(19) United States
(12) Patent Application Publication
(10) Pub. No.: US 2026/0369348 A1
(43) Pub. Date: Nov. 19, 2026

(54) METHODS AND SYSTEMS FOR MUTATION SIGNATURE ATTRIBUTION

(71) Applicant: Foundation Medicine, Inc., Boston, MA (US)

(72) Inventors: Zoe R. FLEISCHMANN, Jamaica Plain, MA (US); Ethan S. SOKOL, Somerville, MA (US); Brennan DECKER, Winchester, MA (US); Douglas A. MATA, Cambridge, MA (US); Smruthy K. SIVAKUMAR, Everett, MA (US)

(73) Assignee: Foundation Medicine, Inc., Boston, MA (US)

(21) Appl. No.: 19/133,491

(22) PCT Filed: Nov. 28, 2023
(86) PCT No.: PCT/US2023/081331

§ 371 Date: May 28, 2025

Related U.S. Application Data
(60) Provisional application No. 63/428,681, filed on Nov. 29, 2022.

(51) Int. Cl. G16B 20/20; C12Q 1/6855; C12Q 1/6874; G16B 20/10; G16B 40/20; G16B 40/30

(52) U.S. Cl. CPC G16B 20/20; C12Q 1/6855; C12Q 1/6874; G16B 20/10; G16B 40/20; G16B 40/30

(57) ABSTRACT

Methods for identifying mutational signatures associated with a sample from an individual are described. The methods may comprise, for example, receiving, using one or more processors, sequence read data associated with the sample; determining, using the one or more processors, a mutational profile of the sample based on the sequence read data; inputting, using the one or more processors, the mutational profile into an autoencoder model, wherein the autoencoder model is trained using training data related to a plurality of mutational signatures; and predicting, using the one or more processors, one or more mutational signatures of the plurality of mutational signatures associated with the sample based on an output of the autoencoder model. In some embodiments, the output of the autoencoder model is associated with a dimensionality value, and wherein the dimensionality value is less than a number of the plurality of mutational signatures.

### CROSS-REFERENCE TO RELATED APPLICAITONS

This application claims the priority benefit of U.S. Provisional Patent Application Ser. No. 63/428,681, filed Nov. 29, 2022, the contents of which are incorporated herein by reference in their entirety.

### FIELD OF THE DISCLOSURE

The present disclosure relates generally to methods and systems for analyzing genomic profiling data, and more specifically to methods and systems for identifying a mutation signature using genomic profiling data.

### BACKGROUND

Mutational processes are present in many cancers and underly much of tumorigenesis and evolution. Certain characteristic patterns of mutations, e.g., mutational signatures, have been identified and associated with various risk factors for cancer. Detecting these mutational signatures has largely been based on whole exome sequencing (WES) and non-negative matrix factorization (NNMF) based identification of signatures. However, for samples with fewer mutations and/or data derived from targeted sequencing, mutational calling can be challenging. Existing methods perform poorly with data comprising sparse matrices of mutations, which is typical of samples with fewer mutations or from samples processed via targeted sequencing.

Current methods for analyzing mutational signatures (both short variant and copy number) use non-negative matrix factorization (NNMF). This method has several limitations—for example, the result is highly dependent on the number of mutational signatures, a parameter that must be declared a priori. For example, UV exposure is associated with two mutational signatures in the current set of short variant reference signatures. In some instances, the number of mutational signatures discovered can be artificially inflated by increasing this parameter. Additionally, NNMF represents each signature as static. While a static signature can be helpful for interpretation, there is a lot of stochasticity in how exposures manifest, and this is not captured using existing methods.

Another limitation with current methods for analyzing mutational signatures is the uncertainty associated with attributing signatures in a new sample. Currently, regression methods are used to determine likely exposures for a given sample. This method, however, has a particularly high false negative rate. For example, Tobacco is thought to cause 80-90% of lung cancer (Centers for Disease Control and Prevention), but a tobacco signature is detected in approximately 28% of non-small cell lung cancer (NSCLC) samples. This false negative rate is further exacerbated by the problem explained in the previous paragraph: mutagenic exposures have complex signatures and are non-deterministic processes.

Additionally, current methods perform poorly for targeted gene panel sequencing tests, where the sparsity of feature data limits the utility of information obtained. For example, current methods require detection of at least 20 signature assessable mutations per sample for robust short variant signature prediction. In other words, samples with a low tumor mutational burden (e.g., low number of mutations) will not provide reliable results if analyzed under current methods. This limits the number of samples analyzed using targeted gene panel sequencing tests that can be used for signature calling.

Accordingly, there is a need for a method to reliably identify a mutation signature in a variety of samples and in samples processed using a variety of sequencing techniques, particularly for samples and sequencing techniques associated with sparse mutational data.

### BRIEF SUMMARY

Embodiments of the present disclosure provide methods for the analysis, discovery, and attribution of mutational signatures using genomic data that address the limitations of the current methods for identifying mutational signatures. Embodiments of the present disclosure use an autoencoder model, which is a neural network constructed from two parts: an encoder and a decoder, to analyze, discover, and/or attribute mutational signatures based on mutational profiles derived from sequencing data. The encoder projects higher dimensional input data to a lower dimensional space (e.g., latent space data), while the decoder uses the latent space data to reconstruct the input data.

Embodiments of the present disclosure can receive a mutational profile of one or more samples as input to the autoencoder model. The encoder portion of the autoencoder model can compress this input into latent space data. The autoencoder model can use this latent space data to identify one or more mutational signatures associated with the mutational profile. For identification of mutational signatures, decreasing to the lower dimensional space forces the algorithm to find consistent patterns (i.e., signatures) in the input data. By changing the dimensionality of the lower dimensional space, the algorithm may be tuned for applications including, but not limited to, imputing the full mutation profile of the sample, de novo signature discovery, error or noise correction, and signature attribution in new samples. Accordingly, embodiments of the present disclosure provide systems and methods that can be used to discover new mutational signatures and attribute these or other mutational signatures in new samples. Embodiments of the present disclosure overcome the limitations of the prior methods discussed above. For example, the methods disclosed herein can provide robust and reliable mutational signature identification for a variety of samples, including samples with low mutation counts, lower tumor mutational burdens, and the like. The methods disclosed herein are thus also more flexible and versatile than current methods. Embodiments of the present disclosure can further impute a full mutation profile from a subset of whole genome or whole exome sequencing data (e.g., from sequencing data provided by a panel sequencing test) and may also be used for germline filtering or error correction.

Embodiments of the present disclosure comprise systems and methods for identifying mutational signatures associated with a sample from a sample. In one or more embodiments, the method can comprise providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; determining, using the one or more processors, a mutational profile of the sample based on the sequence read data; inputting, using the one or more processors, the mutational profile into an autoencoder model, wherein the autoencoder model is trained using training data related to a plurality of mutational signatures; and predicting, using the one or more processors, one or more mutational signatures of the plurality of mutational signatures associated with the sample based on an output of the autoencoder model, wherein the output of the autoencoder model is associated with a dimensionality value, and wherein the dimensionality value is less than a number of the plurality of mutational signatures.

In some embodiments, the autoencoder model comprises multiple layers. In some embodiments, the plurality of mutational signatures corresponds to a plurality of predefined mutational signatures. In some embodiments, the plurality of mutational signatures corresponds to a plurality of COSMIC mutational signatures. In some embodiments, the plurality of predefined mutational signatures comprise a DNA mismatch repair (MMR) signature, a tobacco signature, a UV signature, a putative polymerase epsilon (POLE) signature, an apolipoprotein B mRNA-editing enzyme, catalytic polypeptide (APOBEC) signature, an alkylating agent resistance signature, or a combination thereof.

In some embodiments, predicting the one or more mutational signatures comprises inputting the output of the autoencoder model into a statistical model, the statistical model comprising at least one of a random forest model, a gradient boosting model, a logistic regression model, a support vector machine (SVM), a decision tree model, a density-based spatial clustering of applications with noise (DBSCAN), ordering points to identify the clustering structure (OPTICS); a gaussian mixture model (GMM), a k-means clustering model, a k nearest neighbors model, or a hierarchical clustering model. In such embodiments, the method further comprises training the statistical model, wherein the statistical model comprises a classifier model, wherein training the classifier model comprises: inputting, using the one or more processors, a plurality of training mutational profiles into the autoencoder model; inputting, using the one or more processors, outputs of the autoencoder model corresponding to the plurality of training mutational profiles into an unlabeled classifier model; and identifying, using the one or more processors, one or more mutational signatures based on clustering of the outputs of the autoencoder model.

In some embodiments, the mutational profile comprises single-base substitutions. In some embodiments, the subject is suspected of having or is determined to have cancer. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17 p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the method further comprises treating the subject with an anti-cancer therapy.

In some embodiments, the anti-cancer therapy comprises a targeted anti-cancer therapy. In some embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In such embodiments, the one or more bait molecules comprise one or more nucleic acid molecules, each comprising a region that is complementary to a region of a captured nucleic acid molecule.

In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In such embodiments, the sequencing comprises massively parallel sequencing, and the massively parallel sequencing technique comprises next generation sequencing (NGS).

In some embodiments, the sequencer comprises a next generation sequencer. In some embodiments, one or more of the plurality of sequencing reads overlap one or more gene loci within one or more subgenomic intervals in the sample. In such embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In some embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-16, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In some embodiments, the method further comprises generating, by the one or more processors, a report indicating the one or more predicted mutational signatures. In such embodiments, the method further comprises transmitting the report to a healthcare provider. In some embodiments, the report is transmitted via a computer network or a peer-to-peer connection.

Embodiments in accordance with this disclosure comprise methods for identifying mutational signatures associated with a sample from an individual. In some embodiments, methods comprise receiving, using one or more processors, sequence read data associated with the sample; determining, using the one or more processors, a mutational profile of the sample based on the sequence read data; inputting, using the one or more processors, the mutational profile into an encoder model, wherein the encoder model is trained using training data related to a plurality of mutational signatures; and predicting, using the one or more processors, one or more mutational signatures of the plurality of mutational signatures associated with the sample based on an output of the encoder model, wherein the output of the encoder model is associated with a dimensionality value, and wherein the dimensionality value is less than a number of the plurality of mutational signatures.

In some embodiments, methods comprise receiving, using one or more processors, sequence read data associated with the sample; determining, using the one or more processors, a mutational profile of the sample based on the sequence read data; inputting, using the one or more processors, the mutational profile into an encoder model, wherein the encoder model is trained using training data related to a plurality of mutational signatures; and predicting, using the one or more processors, one or more mutational signatures of the plurality of mutational signatures associated with the sample based on an output of the encoder model, wherein the output of the encoder model is associated with a dimensionality value, and wherein the dimensionality value is less than or equal to a number of the plurality of mutational signatures.

In some embodiments, the encoder model comprises multiple layers. In some embodiments, the plurality of mutational signatures corresponds to a plurality of predefined mutational signatures. In some embodiments, the plurality of mutational signatures corresponds to a plurality of COSMIC mutational signatures. In some embodiments, the plurality of predefined mutational signatures comprise a DNA mismatch repair (MMR) signature, a tobacco signature, a UV signature, a putative polymerase epsilon (POLE) signature, an apolipoprotein B mRNA-editing enzyme, catalytic polypeptide (APOBEC) signature, an alkylating agent resistance signature, or a combination thereof.

In some embodiments, predicting the one or more mutational signatures comprises inputting the output of the encoder model into a statistical model. In some embodiments, the statistical model comprises at least one of a random forest model, a gradient boosting model, a logistic regression model, a support vector machine (SVM), a decision tree model, a density-based spatial clustering of applications with noise (DBSCAN), ordering points to identify the clustering structure (OPTICS); a gaussian mixture model (GMM), a k-means clustering model, a k nearest neighbors model, or a hierarchical clustering model. In such embodiments the method further comprises training the statistical model, wherein the statistical model is a classifier model, wherein training the statistical model comprises: inputting, using the one or more processors, a plurality of training mutational profiles into the encoder model; inputting, using the one or more processors, outputs of the encoder model corresponding to the plurality of training mutational profiles into an unlabeled classifier model; and identifying, using the one or more processors, one or more mutational signatures based on clustering of the outputs of the encoder model.

In some embodiments, the mutational profile comprises less than twenty mutations. In such embodiments, the mutational profile comprises at least three mutations. In some embodiments, the mutational profile is based on a targeted sequencing panel or a targeted-exome sequencing panel. In some embodiments, the mutational profile is based on a whole exome sequencing technique or a whole genome sequencing technique. In some embodiments, the sequence read data associated with the sample is derived from a single biopsy sample. In some embodiments, the sequence read data associated with the sample is derived from multiple biopsy samples. In some embodiments, the sequence read data associated with the sample is derived from single cell sequencing. In some embodiments, the sequence read data associated with the sample is derived from circulating tumor DNA in a liquid biopsy sample. In some embodiments, the sequence read data associated with the sample is derived from RNA in a liquid biopsy sample.

In some embodiments, the mutational profile comprises single-base substitutions. In some embodiments, the mutational profile comprises double-base substitutions, insertions, deletions, copy number, rearrangement information, or a combination thereof.

In some embodiments, the training data comprises training mutational profiles comprising single-base substitutions, double-base substitutions, insertions, deletions, copy number, rearrangement information, or a combination thereof.

In some embodiments, the encoder model is trained by: receiving, using the one or more processors, the training data based on a plurality of training samples; and training, using the one or more processors, an autoencoder model comprising the encoder model based on the training data to obtain a trained autoencoder model. In such embodiments, training the autoencoder model comprises: inputting, using the one or more processors, the training data into an untrained encoder model; inputting, using the one or more processors, an output of the untrained encoder model into a decoder model of the autoencoder model; determining, using the one or more processors, a reconstruction value of an output of the decoder model based on the training data and the output of the decoder model; and updating, using the one or more processors, the untrained encoder model based on the reconstruction value.

In some embodiments, the training data corresponds to a sequence length of 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, or a combination thereof. In some embodiments, the training data corresponds to a sequence length less than 0.5 Megabases. In some embodiments, the training data corresponds to a sequence length greater than 2.0 Megabases. In some embodiments, the training data comprises germline alterations and somatic alterations and the reconstruction value is based on the somatic alterations.

In some embodiments, the method further comprises determining a number of mutations present in a training sample of the plurality of training samples; and removing the training sample from the training data if the number of mutations present in the training sample is below a predetermined threshold. In such embodiments, the predetermined threshold is greater than three.

In some embodiments, the method further comprises validating the trained autoencoder model, the validating comprising: obtaining, using the one or more processors, validation data based on the plurality of training samples, wherein the training data comprises the validation data; and inputting, using the one or more processors, the validation data into a trained encoder model of the trained autoencoder model; inputting, using the one or more processors, an output of the trained encoder model into the decoder model; determining, using the one or more processors, a reconstruction value of a validation output of the decoder model based on the validation data; and updating, using the one or more processors, the encoder model based on the reconstruction value.

In some embodiments, the method further comprise validating the trained autoencoder model, the validating comprising: obtaining, using the one or more processors, validation data based on the plurality of training samples, wherein the training data comprises the validation data; and inputting, using the one or more processors, the validation data into a trained encoder model of the trained autoencoder model; inputting, using the one or more processors, an output of the trained encoder model into the decoder model; determining, using the one or more processors, a concordance value between the training data and a validation output of the decoder model; and updating, using the one or more processors, the encoder model based on the concordance value.

In some embodiments, obtaining the validation data comprises: receiving, by the one or more processors, raw validation data; down sampling, using the one or more processors, the raw validation data to obtain the validation data, and wherein the reconstruction value or the concordance value is determined based on the raw validation data. In such embodiments, determining the reconstruction value is based on the somatic alterations comprising the raw validation data and the somatic alterations comprising the validation data.

In some embodiments, the validation data is associated with sequence lengths of 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, or a combination thereof. In some embodiments, the training data corresponds to a sequence length less than 0.5 Megabases. In some embodiments, the training data corresponds to a sequence length greater than 2.0 Megabases.

In some embodiments, the method further comprises assigning, using the one or more processors, a therapy for the individual based on the one or more mutational signatures. In some embodiments, the method further comprises administering, using the one or more processors, a treatment to the individual based on the one or more mutational signatures. In some embodiments, the method further comprises associating, using the one or more processors, the individual with a clinical trial based on the one or more mutational signatures. In some embodiments, the method further comprises monitoring, using the one or more processors, a prognosis of the individual based on the one or more mutational signatures. In some embodiments, the method further comprises predicting, using the one or more processors, one or more clinical outcomes based on the one or more mutational signatures.

Embodiments of the present disclosure provide methods for diagnosing a disease. The method can comprise: diagnosing that an individual has a disease based on a determination of a mutational signature associated with a sample from the individual, wherein the mutational signature is determined according to the methods described above.

Embodiments of the present disclosure provide methods for selecting an anti-cancer therapy, the method comprises: responsive to determining a mutational signature associated with sample from an individual, selecting an anti-cancer therapy for the individual, wherein mutational signature is determined according to the methods described above.

Embodiments of the present disclosure provide methods for treating a cancer in a individual, comprising: responsive to determining a mutational signature associated with a sample from an individual, administering an effective amount of an anti-cancer therapy to the individual, wherein the mutational signature is determined according to the methods described above.

Embodiments of the present disclosure provide methods for monitoring cancer progression or recurrence in an individual, the method comprising: determining a first mutational signature in a first sample obtained from the individual at a first time point according to the methods described above; determining a second mutational signature in a second sample obtained from the individual at a second time point; and comparing the first mutational signature to the second mutational signature, thereby monitoring the cancer progression or recurrence.

In some embodiments, the second mutational signature for the second sample is determined according to the method of any one of the embodiments above. In some embodiments, the method further comprises selecting an anti-cancer therapy for the individual in response to the cancer progression. In some embodiments, the method further comprises administering an anti-cancer therapy to the individual in response to the cancer progression. In some embodiments, the method further comprises adjusting an anti-cancer therapy for the individual in response to the cancer progression. In some embodiments, the method further comprises adjusting a dosage of the anti-cancer therapy or selecting a different anti-cancer therapy in response to the cancer progression. In some embodiments, the method further comprises administering the adjusted anti-cancer therapy to the individual.

In some embodiments, the first time point is before the individual has been administered an anti-cancer therapy, and wherein the second time point is after the individual has been administered the anti-cancer therapy. In some embodiments, the individual has a cancer, is at risk of having a cancer, is being routine tested for cancer, or is suspected of having a cancer.

In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments of the methods described above, the method can further comprise determining, identifying, or applying the mutational signature of the sample as a diagnostic value associated with the sample. In some embodiments, the method can further comprise generating a genomic profile for the individual based on the determination of the mutational signature. In some embodiments, the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the individual further comprises results from a nucleic acid sequencing-based test. In some embodiments, the method further comprises selecting an anti-cancer therapy, administering an anti-cancer therapy, or applying an anti-cancer therapy to the individual based on the generated genomic profile.

In some embodiments of the methods described above, the determination of the mutational signature associated with the sample is used in making suggested treatment decisions for the individual. In some embodiments of the methods described above, the determination of the mutational signature associated with the sample is used in applying or administering a treatment to the individual.

Embodiments of the present disclosure further comprise systems for identifying mutational signatures associated with a sample from a sample. In some embodiments, the systems can comprise one or more processors and a memory communicatively coupled to the one or more processors. The memory can be configured to store instructions that, when executed by the one or more processors, cause the system to perform a method comprising: receiving, using the one or more processors, sequence read data associated with the sample; determining, using the one or more processors, a mutational profile of the sample based on the sequence read data; inputting, using the one or more processors, the mutational profile into an encoder model, wherein the encoder model is trained using training data related to a plurality of mutational signatures; and predicting, using the one or more processors, one or more mutational signatures of the plurality of mutational signatures associated with the sample based on an output of the encoder model, wherein the output of the encoder model is associated with a dimensionality value, and wherein the dimensionality value is less than a number of the plurality of mutational signatures.

Embodiments of the present disclosure further comprise a non-transitory computer-readable storage medium for identifying mutational signatures associated with a sample from a sample. In some embodiments, the non-transitory computer-readable storage medium stores one or more programs, the one or more programs comprising instructions, which when executed by one or more processors of a system, cause the system to perform a method comprising: receiving, using the one or more processors, sequence read data associated with the sample; determining, using the one or more processors, a mutational profile of the sample based on the sequence read data; inputting, using the one or more processors, the mutational profile into an encoder model, wherein the encoder model is trained using training data related to a plurality of mutational signatures; and predicting, using the one or more processors, one or more mutational signatures of the plurality of mutational signatures associated with the sample based on an output of the encoder model, wherein the output of the encoder model is associated with a dimensionality value, and wherein the dimensionality value is less than a number of the plurality of mutational signatures.

Embodiments of the present disclosure further comprise systems and methods for identifying mutational signatures. In some embodiments, the method comprises: receiving, using one or more processors, sequence read data associated with a plurality of samples; determining, using the one or more processors, mutational profiles of the plurality of samples based on the sequence read data; inputting, using the one or more processors, the mutational profiles into an encoder model; and inputting, using the one or more processors, outputs of the encoder model corresponding to the mutational profiles into an unsupervised machine learning model; and identifying, using the one or more processors, one or more mutational signatures based on clustering of the outputs of the encoder model.

In some embodiments, the unsupervised machine learning model comprises at least one of a density-based spatial clustering of applications with noise (DBSCAN), ordering points to identify the clustering structure (OPTICS); a gaussian mixture model (GMM), a k-means clustering model, a k nearest neighbors model, or a hierarchical clustering model. In some embodiments, a mutational profile of the mutational profiles comprise at least three mutations. In some embodiments, the mutational profiles are based on a targeted sequencing panel or a targeted-exome sequencing panel, a whole exome sequencing technique or a whole genome sequencing technique. In some embodiments, an output of the outputs of the encoder model is associated with a dimensionality value, and wherein the dimensionality value is less than a number of a plurality of mutational signatures used to obtain the output.

In some embodiments, the sequence read data associated with the plurality of samples is derived from single cell sequencing. In some embodiments, the sequence read data associated with the plurality of samples is derived from circulating tumor DNA in a liquid biopsy sample. In some embodiments, the sequence read data associated with the plurality of samples is derived from RNA in a liquid biopsy sample. In some embodiments, the mutational profiles comprise single-base substitutions. In some embodiments, the mutational profiles comprise double-base substitutions, insertions, deletions, copy number, rearrangement information, or a combination thereof.

In some embodiments, the encoder model is trained by: receiving, using the one or more processors, the training data based on a plurality of training samples; and training, using the one or more processors, an autoencoder model comprising the encoder model based on the training data to obtain a trained autoencoder model.

In some embodiments, training the autoencoder model comprises: inputting, using the one or more processors, the training data into an untrained encoder model; inputting, using the one or more processors, an output of the untrained encoder model into a decoder model of the autoencoder model; determining, using the one or more processors, a reconstruction value of an output of the decoder model based on the training data and the output of the decoder model; and updating, using the one or more processors, the untrained encoder model based on the reconstruction value to produce a trained encoder model.

In some embodiments, the training data comprises training mutational profiles comprising single-base substitutions, double-base substitutions, insertions, deletions, or a combination thereof. In some embodiments, the training data corresponds to a sequence length of 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, or a combination thereof. In some embodiments, the training data corresponds to a sequence length less than 0.5 Megabases. In some embodiments, the training data corresponds to a sequence length greater than 2.0 Megabases.

In some embodiments, the method further comprises determining a number of mutations present in a training sample of the plurality of training samples; removing the training sample from the training data if the number of mutations present in the training sample is below a predetermined threshold. In some embodiments, the predetermined threshold is greater than three.

In some embodiments, the method further comprises validating the trained autoencoder model, the validating comprising: obtaining, using the one or more processors, validation data based on the plurality of training samples, wherein the training data comprises the validation data; inputting, using the one or more processors, the validation data into the trained encoder model of the trained autoencoder model; inputting, using the one or more processors, an output of the trained encoder model into the decoder model; determining, using the one or more processors, a reconstruction value of a validation output of the decoder model based on the validation data; and updating, using the one or more processors, the encoder model based on the reconstruction value.

In some embodiments, the method further comprises validating the trained autoencoder model, the validating comprising: obtaining, using the one or more processors, validation data based on the plurality of training samples, wherein the training data comprises the validation data; inputting, using the one or more processors, the validation data into the trained encoder model; inputting, using the one or more processors, an output of the trained encoder model into the decoder model; determining, using the one or more processors, a concordance value between the training data and an output of the decoder model; and updating, using the one or more processors, the encoder model based on the concordance value.

In some embodiments, obtaining the validation data comprises: receiving, by the one or more processors, raw validation data; down sampling, using the one or more processors, the raw validation data to obtain the validation data. In some embodiments, the raw validation data comprises germline alterations and somatic alterations and the validation data comprises the somatic alterations. In some embodiments, determining the reconstruction value is based on the somatic alterations comprising the raw validation data and the somatic alterations comprising the validation data.

In some embodiments, the validation data is associated with sequence lengths of 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, or a combination thereof. In some embodiments, the training data corresponds to a sequence length less than 0.5 Megabases. In some embodiments, the training data corresponds to a sequence length greater than 2.0 Megabases.

In some embodiments, the method further comprises assigning, using the one or more processors, a therapy for the individual based on the one or more mutational signatures. In some embodiments, the method further comprises administering, using the one or more processors, a treatment to the individual based on the one or more mutational signatures. In some embodiments, the method further comprises associating, using the one or more processors, the individual with a clinical trial based on the one or more mutational signatures. In some embodiments, the method further comprises monitoring, using the one or more processors, a prognosis of the individual based on the one or more mutational signatures. In some embodiments, the method further comprises predicting, using the one or more processors, one or more clinical outcomes based on the one or more mutational signatures.

Embodiments, of the present disclosure further comprise methods for diagnosing a disease, the methods comprising: diagnosing that an individual has a disease based on a determination of a mutational signature associated with a sample from the individual, wherein the mutational signature is determined according to the methods described above.

Embodiments, of the present disclosure further comprise methods for selecting an anti-cancer therapy, the method comprising: responsive to determining a mutational signature associated with sample from an individual, selecting an anti-cancer therapy for the individual, wherein mutational signature is determined according to the methods described above.

Embodiments, of the present disclosure further comprise methods for treating a cancer in an individual, comprising: responsive to determining a mutational signature associated with a sample from an individual, administering an effective amount of an anti-cancer therapy to the individual, wherein the mutational signature is determined according to the methods described above.

Embodiments, of the present disclosure further comprise methods for monitoring cancer progression or recurrence in an individual, the method comprising: determining a first mutational signature in a first sample obtained from the individual at a first time point according to the methods described above; determining a second mutational signature in a second sample obtained from the individual at a second time point; and comparing the first mutational signature to the second mutational signature, thereby monitoring the cancer progression or recurrence.

In some embodiments, the second mutational signature for the second sample is determined according to the methods described above. In some embodiments, the method further comprises selecting an anti-cancer therapy for the individual in response to the cancer progression. In some embodiments, the method further comprises administering an anti-cancer therapy to the individual in response to the cancer progression. In some embodiments, the method further comprises adjusting an anti-cancer therapy for the individual in response to the cancer progression. In some embodiments, the method further comprises adjusting a dosage of the anti-cancer therapy or selecting a different anti-cancer therapy in response to the cancer progression. In some embodiments, the method further comprises administering the adjusted anti-cancer therapy to the individual.

In some embodiments, the first time point is before the individual has been administered an anti-cancer therapy, and wherein the second time point is after the individual has been administered the anti-cancer therapy. In some embodiments, the individual has a cancer, is at risk of having a cancer, is being routine tested for cancer, or is suspected of having a cancer.

In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments of the methods described above, the method can further comprise determining, identifying, or applying the mutational signature of the sample as a diagnostic value associated with the sample. In some embodiments, the method can further comprise generating a genomic profile for the individual based on the determination of the mutational signature. In some embodiments, the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the individual further comprises results from a nucleic acid sequencing-based test. In some embodiments, the method further comprises selecting an anti-cancer therapy, administering an anti-cancer therapy, or applying an anti-cancer therapy to the individual based on the generated genomic profile.

In some embodiments of the methods described above, the determination of the mutational signature associated with the sample is used in making suggested treatment decisions for the individual. In some embodiments of the methods described above, the determination of the mutational signature associated with the sample is used in applying or administering a treatment to the individual.

Embodiments of the present disclosure further comprise systems for identifying mutational signatures. In one or more embodiments, the system can comprise one or more processors and a memory communicatively coupled to the one or more processors. The processor can be configured to store instructions that, when executed by the one or more processors, cause the system to perform a method comprising: receiving, using the one or more processors, sequence read data associated with a plurality of samples; determining, using the one or more processors, mutational profiles of the plurality of samples based on the sequence read data; inputting, using the one or more processors, the mutational profiles into an encoder model; and inputting, using the one or more processors, outputs of the encoder model corresponding to the mutational profiles into an unsupervised machine learning model; and identifying, using the one or more processors, one or more mutational signatures based on clustering of the outputs of the encoder model.

Embodiments of the present disclosure further comprise non-transitory computer-readable storage mediums for identifying mutational signatures. In one or more embodiments, the non-transitory computer-readable storage medium can store one or more programs, the one or more programs comprising instructions, which when executed by one or more processors of a system, cause the system to perform a method comprising: receiving, using the one or more processors, sequence read data associated with a plurality of samples; determining, using the one or more processors, mutational profiles of the plurality of samples based on the sequence read data; inputting, using the one or more processors, the mutational profiles into an encoder model; and inputting, using the one or more processors, outputs of the encoder model corresponding to the mutational profiles into an unsupervised machine learning model; and identifying, using the one or more processors, one or more mutational signatures based on clustering of the outputs of the encoder model.

Embodiments of the present disclosure further comprise methods for training an autoencoder model to predict a mutational profile of a sample, the method comprising: receiving, using one or more processors, training data comprising a plurality of mutational profiles from a plurality of training samples; inputting, using the one or more processors, the plurality of mutational profiles into an encoder model of the autoencoder model, wherein the encoder model is configured to output derived data; inputting the derived data into a decoder model of the autoencoder model, wherein the decoder model is configured to output a reconstruction of the plurality of mutational profiles based on the derived data; receiving a reconstruction value based on a comparison of the training data and the output of the decoder model; and updating the encoder model based on the reconstruction value to produce a trained encoder model.

In some embodiments, the method further comprises validating the trained autoencoder model, the validating comprising: obtaining, using the one or more processors, validation data based on the plurality of training samples; and inputting, using the one or more processors, the validation data into the trained encoder model of the autoencoder model; inputting, using the one or more processors, an output of the trained encoder model into the decoder model; determining, using the one or more processors, a reconstruction value of an output of the decoder model based on the validation data; and updating, using the one or more processors, the encoder model based on the reconstruction value.

In some embodiments, the method further comprises validating the trained autoencoder model, the validating comprising: obtaining, using the one or more processors, validation data based on the plurality of training samples; and inputting, using the one or more processors, the validation data into the trained encoder model; inputting, using the one or more processors, an output of the trained encoder model into the decoder model; determining, using the one or more processors, a concordance value between the training data and an output of the decoder model; and updating, using the one or more processors, the encoder model based on the concordance value.

In some embodiments, obtaining the validation data comprises: receiving, by the one or more processors, raw validation data corresponding to one or more mutational profiles; down sampling, using the one or more processors, the raw validation data to obtain the validation data, wherein the reconstruction value or the concordance value is determined based on the raw validation data.

Embodiments of the present disclosure further provide systems and methods for determining an alkylating agent resistance signature status of a sample from a subject. In some embodiments the method comprises: receiving, using one or more processors, sequence read data associated with a sample from the subject; selecting, using the one or more processors, a plurality of reads from the sequence read data; determining, using the one or more processors, an alkylating agent resistance profile based on the selected plurality of reads; inputting, using the one or more processors, the alkylating agent resistance profile into a statistical model; and predicting, using the one or more processors, the alkylating agent resistance signature status based on an output of the statistical model.

Embodiments of the present disclosure further provide methods of treating a subject having a cancer, the method comprising: determining an alkylating agent resistance signature status of a sample from the subject; and treating the subject with an alkylating agent if the subject is determined to have a non-positive alkylating agent resistance signature status. In some embodiments, determining an alkylating agent resistance signature status is based on the methods described above.

In some embodiments, the subject was previously treated with one or more alkylating agents. In some embodiments, the method further comprises determining a tumor mutation burden (TMB) in the sample from the subject. In some embodiments, the statistical model comprises an encoder model as described above.

In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs), cell-free DNA (cfDNA), circulating tumor DNA (ctDNA), or any combination thereof.

In some embodiments, the sequence read data associated with the sample is derived from RNA in a liquid biopsy sample. In some embodiments, the alkylating agent resistance profile comprises single-base substitutions. In some embodiments, the alkylating agent resistance profile comprises double-base substitutions, insertions, deletions, copy number, rearrangement information, or a combination thereof.

In some embodiments, the method further comprises determining, based on the selected plurality of reads, a MSH6 alteration, a MSH2 alteration, a MLH1 alteration, a PIK3CA alteration, a TP53 alteration, or a combination thereof. In some embodiments, the cancer is a brain cancer, a breast cancer, a colorectal cancer (CRC), a prostate cancer, a non-small cell lung cancer (NSCLC), a neuroendocrine cancer, or a combination thereof.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of an autoencoder model, in accordance with some embodiments of the present disclosure.

FIG. 2 provides a non-limiting example of a process for predicting mutational signatures in a sample, in accordance with some embodiments of the present disclosure.

FIG. 3 provides a non-limiting example of a mutational profile, in accordance with some embodiments of the present disclosure.

FIG. 4 provides a non-limiting example of a block diagram associated with a process for predicting mutational signatures in a sample, in accordance with some embodiments of the present disclosure.

FIG. 5 provides a non-limiting example of a block diagram associated with a process for predicting mutational signatures in a sample, in accordance with some embodiments of the present disclosure.

FIG. 6 provides a non-limiting example of a process for identifying mutational signatures, in accordance with some embodiments of the present disclosure.

FIG. 7 provides a non-limiting example of a block diagram associated with a process for identifying mutational signatures, in accordance with some embodiments of the present disclosure.

FIG. 8 provides a non-limiting example of a process for training a statistical model, in accordance with some embodiments of the present disclosure.

FIG. 9 provides a non-limiting example of a block diagram associated with a process for training a statistical model, in accordance with some embodiments of the present disclosure.

FIG. 10 provides non-limiting examples of mutational profiles, in accordance with some embodiments of the present disclosure.

FIG. 11 provides a non-limiting example of a process for validating a statistical model, in accordance with some embodiments of the present disclosure.

FIG. 12 provides a non-limiting example of a block diagram associated with a process for validating a statistical model, in accordance with some embodiments of the present disclosure.

FIG. 13 provides non-limiting examples of mutational profiles, in accordance with some embodiments of the present disclosure.

FIG. 14 provides a non-limiting example of a block diagram associated with a process for training a statistical model, in accordance with some embodiments of the present disclosure.

FIG. 15 provides a non-limiting example of an output of a statistical model, in accordance with some embodiments of the present disclosure.

FIG. 16 depicts an exemplary computing device or system in accordance with one embodiment of the present disclosure.

FIG. 17 depicts an exemplary computer system or computer network, in accordance with some instances of the systems described herein.

FIG. 18 provides a non-limiting example of a statistical model, in accordance with some embodiments of the present disclosure.

### DETAILED DESCRIPTION

Embodiments of the present disclosure provide methods for the analysis, discovery, and attribution of mutational signatures using genomic data that address the limitations of the current methods for identifying mutational signatures. Embodiments of the present disclosure use an autoencoder model, which is a neural network constructed from two parts: an encoder and a decoder, to analyze, discover, and/or attribute mutational signatures based on mutational profiles derived from sequencing data. The encoder projects higher dimensional input data to a lower dimensional space (e.g., latent space data), while the decoder uses the latent space data to reconstruct the input data.

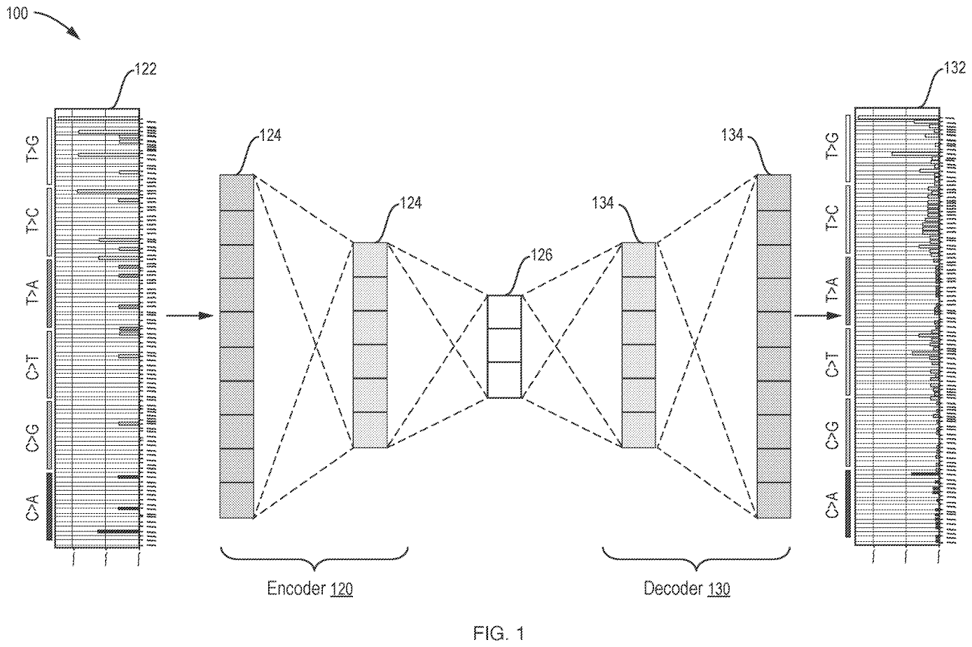

FIG. 1 illustrates an exemplary autoencoder model 100 in accordance with embodiments of the present disclosure. The autoencoder model 100 may be a neural network. The encoder 120 can be trained to receive an input 122 and compress the input 122 via one or more layers 124 to a lower dimensional space, e.g., latent space representation 126. The decoder 130 is trained to do the reverse-decompressing the latent space 126 via one or more layers 134 to output a reconstruction 132 of the input. Thus, the output of the encoder model corresponds to the latent space representation 126, while the output of the decoder model corresponds to the reconstructed input 132. The encoder model 120 and the decoder model 130 can be trained together as described later in the application.

As shown in the figure, embodiments of the present disclosure can receive a mutational profile of one or more samples as an input 122 to the encoder model 120. The encoder model 120 can compress the mutational profile 122 to output a latent space representation 126. The system can use this latent space representation 126 to identify one or more mutational signatures associated with the mutational profile of the sample. For mutational signatures, decreasing to the lower dimensional space forces the algorithm to find consistent patterns (i.e., signatures). By reducing the dimensionality of the mutational profile to the lower dimensional space, the encoder model 120 has applications including but not limited to imputing the full mutation profile of the sample, de novo signature discovery, error or noise correction, and signature attribution in new samples.

Accordingly, embodiments of the present disclosure provide systems and methods that can be used to discover new signatures and attribute these or other signatures in new samples. Embodiments of the present disclosure overcome the limitations the prior methods discussed above. For example, methods disclosed herein can provide robust and reliable mutational signature identification for a variety of samples, including samples with low mutation counts, lower tumor mutational burdens, and the like. In this manner, methods disclosed herein are also more flexible and versatile than current methods. Embodiments of the present disclosure can further impute a full mutation profile from a subset (e.g., from a panel sequencing test), germline filtering or error correction.

In some instances, for example, methods are described that comprise: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; determining, using the one or more processors, a mutational profile of the sample based on the sequence read data; inputting, using the one or more processors, the mutational profile into an autoencoder model, wherein the autoencoder model is trained using training data related to a plurality of mutational signatures; and predicting, using the one or more processors, one or more mutational signatures of the plurality of mutational signatures associated with the sample based on an output of the autoencoder model, wherein the output of the autoencoder model is associated with a dimensionality value, and wherein the dimensionality value is less than a number of the plurality of mutational signatures.

Embodiments in accordance with this disclosure further comprise methods for identifying mutational signatures associated with a sample from an individual. In some embodiments, methods comprise receiving, using one or more processors, sequence read data associated with the sample; determining, using the one or more processors, a mutational profile of the sample based on the sequence read data; inputting, using the one or more processors, the mutational profile into an encoder model, wherein the encoder model is trained using training data related to a plurality of mutational signatures; and predicting, using the one or more processors, one or more mutational signatures of the plurality of mutational signatures associated with the sample based on an output of the encoder model, wherein the output of the encoder model is associated with a dimensionality value, and wherein the dimensionality value is less than a number of the plurality of mutational signatures.

In some embodiments, methods comprise receiving, using one or more processors, sequence read data associated with the sample; determining, using the one or more processors, a mutational profile of the sample based on the sequence read data; inputting, using the one or more processors, the mutational profile into an encoder model, wherein the encoder model is trained using training data related to a plurality of mutational signatures; and predicting, using the one or more processors, one or more mutational signatures of the plurality of mutational signatures associated with the sample based on an output of the encoder model, wherein the output of the encoder model is associated with a dimensionality value, and wherein the dimensionality value is less than or equal to a number of the plurality of mutational signatures.

Embodiments of the present disclosure further comprise systems and methods for identifying mutational signatures. In some embodiments, the method comprises: receiving, using one or more processors, sequence read data associated with a plurality of samples; determining, using the one or more processors, mutational profiles of the plurality of samples based on the sequence read data; inputting, using the one or more processors, the mutational profiles into an encoder model; and inputting, using the one or more processors, outputs of the encoder model corresponding to the mutational profiles into an unsupervised machine learning model; and identifying, using the one or more processors, one or more mutational signatures based on clustering of the outputs of the encoder model.

Embodiments of the present disclosure further comprise methods for training an autoencoder model to predict a mutational profile of a sample, the method comprising: receiving, using one or more processors, training data comprising a plurality of mutational profiles from a plurality of training samples; inputting, using the one or more processors, the plurality of mutational profiles into an encoder model of the autoencoder model, wherein the encoder model is configured to output derived data; inputting the derived data into a decoder model of the autoencoder model, wherein the decoder model is configured to output a reconstruction of the plurality of mutational profiles based on the derived data; receiving a reconstruction value based on a comparison of the training data and the output of the decoder model; and updating the encoder model based on the reconstruction value to produce a trained encoder model.

In some embodiments of the methods described above, the autoencoder model comprises multiple layers. In some embodiments, the plurality of mutational signatures corresponds to a plurality of predefined mutational signatures. In some embodiments, the plurality of mutational signatures corresponds to a plurality of COSMIC mutational signatures. In some embodiments, the plurality of predefined mutational signatures comprise a DNA mismatch repair (MMR) signature, a tobacco signature, a UV signature, a putative polymerase epsilon (POLE) signature, an apolipoprotein B mRNA-editing enzyme, catalytic polypeptide (APOBEC) signature, an alkylating agent resistance signature, or a combination thereof. In some embodiments, the mutational profile comprises single-base substitutions.

In some embodiments, predicting the one or more mutational signatures comprises inputting the output of the autoencoder model into a statistical model, the statistical model comprising at least one of a random forest model, a gradient boosting model, a logistic regression model, a support vector machine (SVM), a decision tree model, a density-based spatial clustering of applications with noise (DBSCAN), ordering points to identify the clustering structure (OPTICS); a gaussian mixture model (GMM), a k-means clustering model, a k nearest neighbors model, or a hierarchical clustering model. In such embodiments, the method further comprises training the statistical model, wherein the statistical model comprises a classifier model, wherein training the classifier model comprises: inputting, using the one or more processors, a plurality of training mutational profiles into the autoencoder model; inputting, using the one or more processors, outputs of the autoencoder model corresponding to the plurality of training mutational profiles into an unlabeled classifier model; and identifying, using the one or more processors, one or more mutational signatures based on clustering of the outputs of the autoencoder model.

Embodiments of the present disclosure further provide systems and methods for determining an alkylating agent resistance signature status of a sample from a subject. In some embodiments the method comprises: receiving, using one or more processors, sequence read data associated with a sample from the subject; selecting, using the one or more processors, a plurality of reads from the sequence read data; determining, using the one or more processors, an alkylating agent resistance profile based on the selected plurality of reads; inputting, using the one or more processors, the alkylating agent resistance profile into a statistical model; and predicting, using the one or more processors, the alkylating agent resistance signature status based on an output of the statistical model.

Accordingly, embodiments of the present disclosure provide systems and methods that can be used to discover new signatures and attribute these or other signatures in new samples. Embodiments of the present disclosure overcome the limitations the prior methods discussed above. For example, methods disclosed herein can provide robust and reliable mutational signature identification for a variety of samples, including samples with low mutation counts, lower tumor mutational burdens, and the like. In this manner, methods disclosed herein are also more flexible and versatile than current methods. Embodiments of the present disclosure can further impute a full mutation profile from a subset (e.g., from a panel sequencing test), germline filtering or error correction.

### Definitions

Unless otherwise defined, all the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The term “mutational signature” is used herein to refer to a pattern of mutations attributed to a common mechanism, e.g., UV radiation, tobacco, aging, etc.

The term “mutational profile” is used herein to refer to the mutations observed in a single sample.

The section headings used herein are for organizational purposes only and are not to be construed as limiting the subject matter described.

### Methods for Identifying Mutational Signatures in a Sample

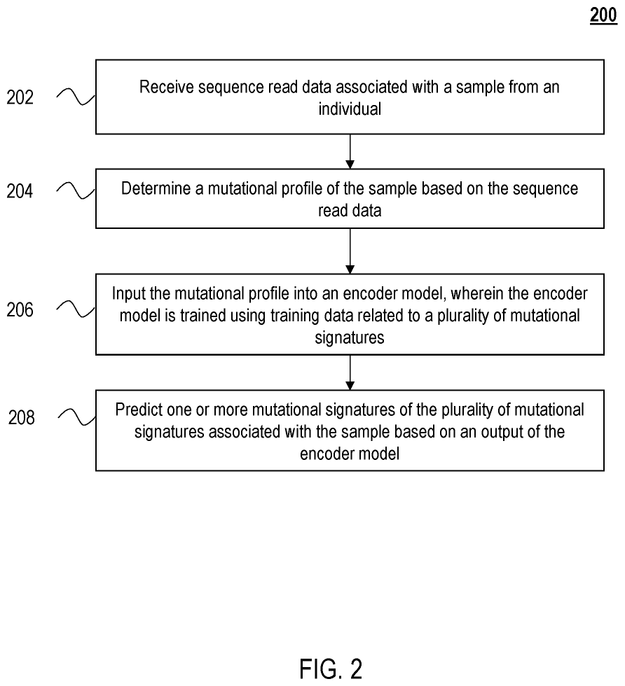

FIG. 2 provides a non-limiting example of a process 200 for identifying mutational signatures associated with a sample from an individual. Process 200 can be performed, for example, using one or more electronic devices implementing a software platform. In some examples, process 200 is performed using a client-server system, and the blocks of process 200 are divided up in any manner between the server and a client device. In other examples, the blocks of process 200 are divided up between the server and multiple client devices. Thus, while portions of process 200 are described herein as being performed by particular devices of a client-server system, it will be appreciated that process 200 is not so limited. In other examples, process 200 is performed using only a client device or only multiple client devices. In process 200, some blocks are, optionally, combined, the order of some blocks is, optionally, changed, and some blocks are, optionally, omitted. In some examples, additional steps may be performed in combination with the process 200. Accordingly, the operations as illustrated (and described in greater detail below) are exemplary by nature and, as such, should not be viewed as limiting.

At block 202 of FIG. 2 the system can receive sequence read data associated with one or more genomic variants in a sample from an individual. In one or more examples, the sample may be a solid biopsy sample or a liquid biopsy sample. In some instances, the sequence read data may be derived from single region sequencing (e.g., sequencing of a single tissue biopsy sample collected from the tumor of the individual). In some instances, the genomic data comprising sequence read data may be derived from multi-region sequencing (e.g., sequencing of multiple tissue biopsy samples collected from the tumor of the individual). In some instances, the genomic data comprising sequence read data may be derived from single cell sequencing data as opposed to bulk tumor sequencing. In some instances, the genomic data comprising sequence read data may be derived from sequencing circulating tumor DNA in a liquid biopsy sample. In some instances, the sequence read data may be derived from RNA in a liquid biopsy sample.

In some instances, the genomic data comprising sequence read data may be derived from targeted sequencing, e.g., targeted exome sequencing. In some instances, the genomic data comprising sequence read data may be derived from broad panel sequencing. In some instances, the sequence read data may be derived from whole genome or whole exome sequencing, e.g., as opposed to targeted exome sequencing or broad panel sequencing to increase the number of genomic features (e.g., the number of short variants, copy number alteration) detected. In one or more examples, the sequence read data may be received by the system as a BAM file.

In one or more examples, the sequence read data may be indicative of a presence or absence of one or more short variants (SVs) in a patient sample. In one or more examples, the sequence read data may also be indicative of the presence or absence of genomic events, such as copy number alterations, rearrangements, insertions, deletions, fusions, chromosomal aneuploidy, whole genome doubling, Catalogue Of Somatic Mutations In Cancer (COSMIC) mutational signatures, microsatellite instability (MSI) status, tumor mutational burden (TMB), or any combination thereof.

At block 204 of FIG. 2, the system can determine a mutational profile of the sample based on the sequence read data. In one or more examples, the system can identify one or more genomic variants in the sample to build a mutational profile.

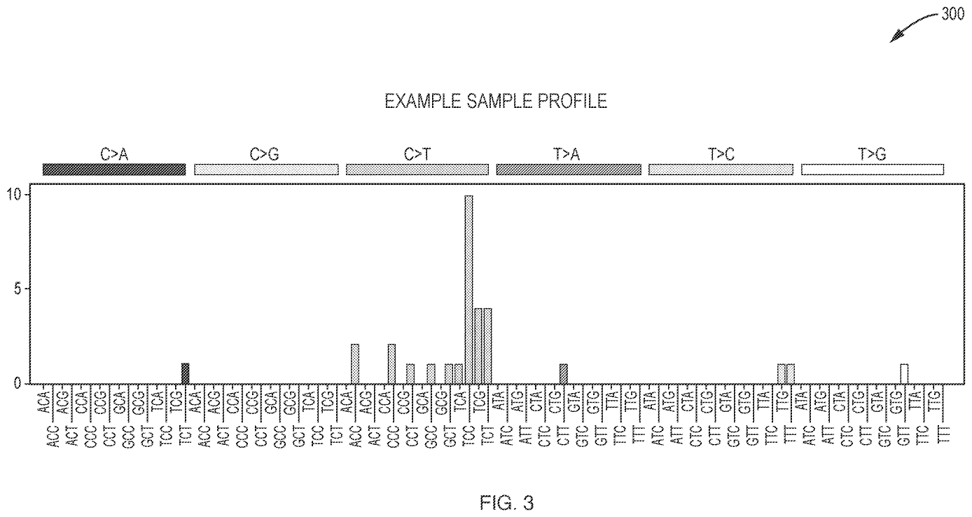

FIG. 3 illustrates a non-limiting example of a mutational profile 300 based on a sample. The mutational profile 300 corresponds to a histogram of single base substitutions identified in the corresponding sample. As shown in the figure, many of the genomic variants (e.g., single-base substitutions) identified in the sample correspond to C to T substitutions. While the mutational profile 300 corresponds to a profile for single base substitution variants, other mutational profiles may be based on other types of alterations without departing from the scope of this disclosure. For example, other mutational profiles could correspond to one or more of single-base substitutions, mutational profile comprises double-base substitutions, insertions, deletions, copy number, and/or rearrangement information.

At block 206 of FIG. 2, the system can input the mutational profile into an encoder model. The encoder model may be trained to identify one or more mutational signatures in a sample based on the mutational profile. In some instances, the encoder model is trained using training data related to a plurality of mutational signatures to identify a plurality of mutational signatures in a sample from an individual. As discussed above regarding FIG. 1, the encoder model may be trained as a part of an autoencoder model comprising an encoder model and a decoder model.

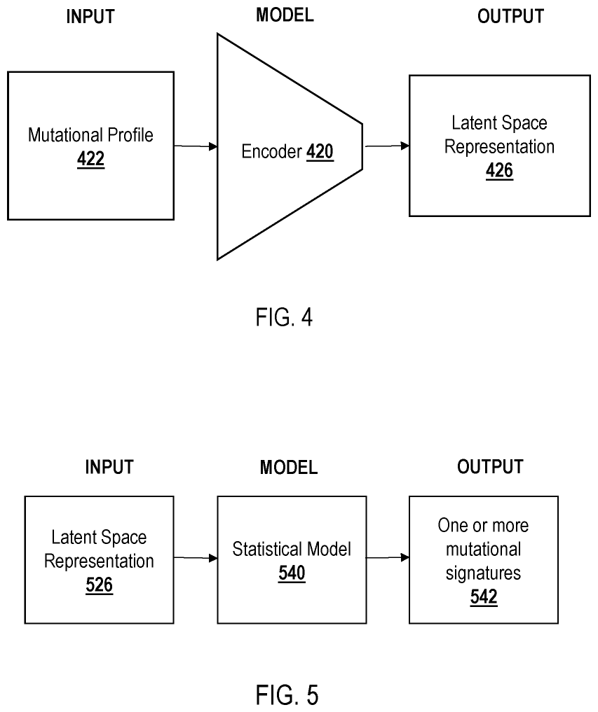

FIG. 4 illustrates an exemplary block diagram 400 corresponding to a process for determining a latent space representation of a mutational profile (e.g., corresponding to block 206). As shown in the figure, the encoder model 420 can receive a mutational profile 422 (e.g., mutational profile 300) as an input. As discussed above, the encoder 420 can be trained to compress the input via one or more neural network layers to a lower dimensional space and output a latent space representation 426 of the mutation profile 422. Compressing the data comprising the mutational profile to the lower dimensional space forces the encoder model 420 to find consistent patterns that can be used to identify mutational signatures associated with the sample.

In some examples, the mutational profile can include at least three mutations. In some examples, the mutational profile can include at least three, five, or seven mutations. The number of mutations in a mutational profile input into the encoder model should have a sufficient number of mutations to determine meaningful patterns and relationships from the data.

In some instances, the output 426 of the encoder model 420 can be associated with a dimensionality value, where this dimensionality value is less than a number of the plurality of mutational signatures. For example, in some embodiments, the encoder may be trained to identify whether a sample is associated with six mutational signatures. In such examples, the encoder model 420 may be trained to produce a latent space with a dimensionality of five or less. In this manner, embodiments of the present disclosure capitalize on the encoder model's ability to identify patterns in the mutational profile by training the encoder to reduce the dimensionality of the latent space to less than the number of mutational signatures.

At block 208 of FIG. 2, the system can predict one or more mutational signatures of the plurality of mutational signatures associated with the sample based on an output of the encoder model (e.g., latent space representation). In some instances, the plurality of mutational signatures may be a plurality of predefined mutational signatures. For example, the plurality of mutational signatures can include, but is not limited to Catalogue of Somatic Mutations in Cancer (COSMIC) mutational signatures, such as a DNA mismatch repair (MMR) signature, a tobacco signature, a UV signature, a putative polymerase epsilon (POLE) signature, an apolipoprotein B mRNA-editing enzyme, an apolipoprotein B mRNA editing enzyme catalytic polypeptide (APOBEC) signature, and/or an alkylating agent resistance signature.

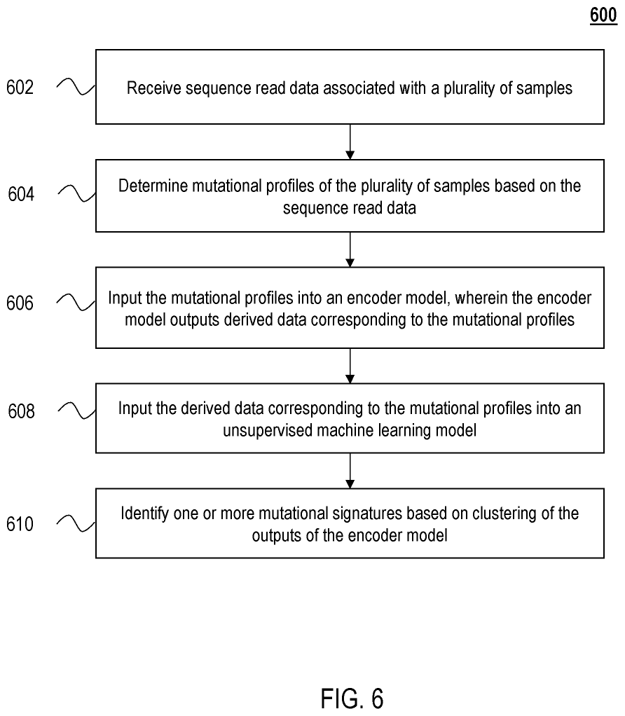

In some instances, the system uses a statistical model (e.g., separate from the autoencoder/encoder model) to predict the one or more mutational signatures. For example, the system can input the latent space representation determined by the encoder model (e.g., encoder model 120, 420) into the statistical model. FIG. 5 illustrates a block diagram for a process 500 for predicting the one or more mutational signatures using a statistical model. As shown in the figure, the37 elinexs 500 includes inputting a latent space representation 526 derived from a mutational profile into a statistical model 540. The statistical model 540 can be trained to predict one or more mutational signatures based on the latent space representation 526 and output the identified mutational signatures 542.

In some instances, the statistical model can correspond to a classifier model. In some instances, the statistical model can correspond to a supervised model such as, but not limited to, a random forest model, a gradient boosting model, a logistic regression model, a support vector machine model, and/or a decision tree model. In some instances, the statistical model can correspond to an unsupervised model such as, but not limited to, a density-based spatial clustering of applications with noise (DBSCAN), ordering points to identify the clustering structure (OPTICS); a gaussian mixture model (GMM), a k-means clustering model, a k nearest neighbors model, and/or a hierarchical clustering model.

### De Novo Signature Identification

Embodiments of the present disclosure can further be used for de novo signature identification in addition to identifying mutational signatures in a sample from an individual. In some instances, the de novo signature identification can be performed based on a plurality of mutational profiles from a plurality of samples. The mutational profiles can be analyzed via an encoder model (e.g., encoder model 120, 420, 520) and a second statistical model to determine the de novo signatures.

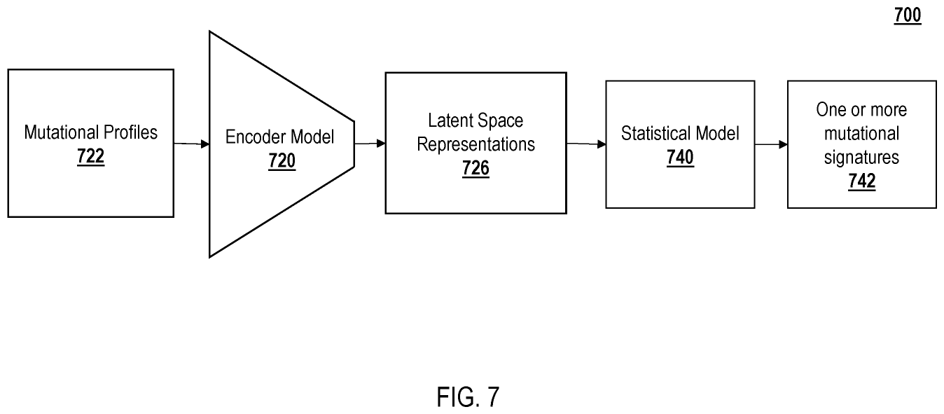

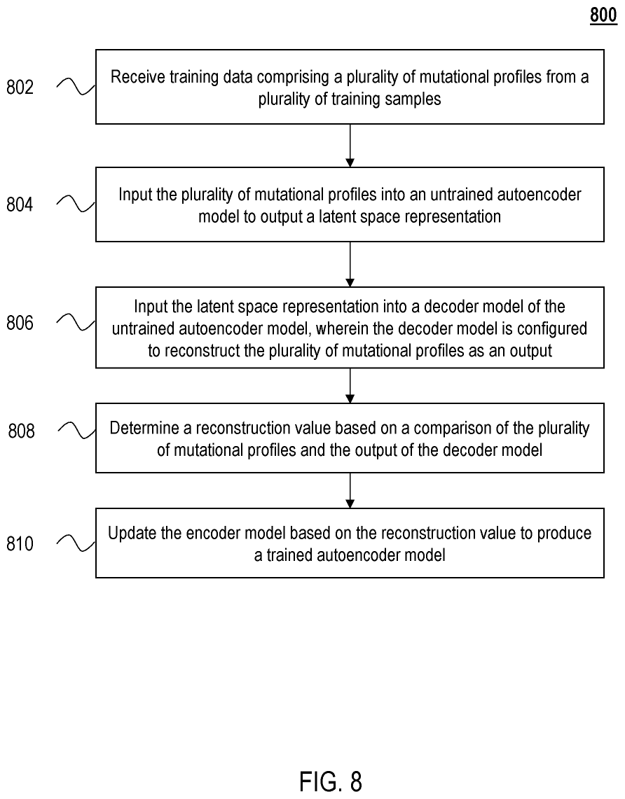

FIG. 6 illustrates an exemplary flow chart of a process 600 for identifying mutational signatures de novo. FIG. 7 illustrates a corresponding block diagram 700 associated with a process for identifying mutational signatures de novo. Process 600 can be performed, for example, using one or more electronic devices implementing a software platform. In some examples, process 600 is performed using a client-server system, and the blocks of process 600 are divided up in any manner between the server and a client device. In other examples, the blocks of process 600 are divided up between the server and multiple client devices. Thus, while portions of process 600 are described herein as being performed by particular devices of a client-server system, it will be appreciated that process 600 is not so limited. In other examples, process 600 is performed using only a client device or only multiple client devices. In process 600, some blocks are, optionally, combined, the order of some blocks is, optionally, changed, and some blocks are, optionally, omitted. In some examples, additional steps may be performed in combination with the process 600. Accordingly, the operations as illustrated (and described in greater detail below) are exemplary by nature and, as such, should not be viewed as limiting.

At block 602 of FIG. 6, the system can receive sequence data associated with a plurality of samples. The plurality of samples can correspond to a plurality of individuals. In one or more examples, the plurality of sample may comprise one or more of solid biopsy samples or liquid biopsy samples.

In some instances, the sequence read data may be derived from single region sequencing (e.g., sequencing of a single tissue biopsy sample collected from the tumor of the individual). In some instances, the genomic data comprising sequence read data may be derived from multi-region sequencing (e.g., sequencing of multiple tissue biopsy samples collected from the tumor of the individual). In some instances, the genomic data comprising sequence read data may be derived from single cell sequencing data as opposed to bulk tumor sequencing. In some instances, the genomic data comprising sequence read data may be derived from sequencing the circulating tumor DNA in a liquid biopsy sample. In some instances, the sequence read data may be derived from RNA in a liquid biopsy sample. In some instances, the sequence read data from different samples may be derived from a mix of sequencing techniques, e.g., some samples may be processed using single region sequencing, some samples may be processed from sequencing circulating tumor DNA in a liquid biopsy sample, etc.

In some instances, the genomic data comprising sequence read data may be derived from targeted sequencing, e.g., targeted exome sequencing. In some instances, the genomic data comprising sequence read data may be derived from broad panel sequencing. In some instances, the sequence read data may be derived from whole genome or whole exome sequencing, e.g., as opposed to targeted exome sequencing or broad panel sequencing to increase the number of genomic features (e.g., the number of short variants, copy number alteration) detected. In some instances, the sequence read data from different samples may be derived from a mix of sequencing techniques, e.g., some samples may be processed using broad panel sequencing, some samples may be processed using targeted panel sequencing, etc. In one or more examples, the sequence read data may be received by the system as a BAM file.

At block 604 of FIG. 6, the system can determine mutational profiles of the plurality of samples based on the sequence read data. For instance, for each sample, the system can identify one or more genomic variants in the sample to build a respective mutational profile, such as the exemplary mutational profile 300 shown in FIG. 3. While the mutational profile 300 corresponds to a profile for single base substitution variants, other mutational profiles may be based on other types of alterations without departing from the scope of this disclosure. For example, other mutational profiles could correspond to one or more of single-base substitutions, mutational profile comprises double-base substitutions, insertions, deletions, copy number, and/or rearrangement information. Block 722 of FIG. 7 corresponds to exemplary mutational profiles 722.

At block 606 of FIG. 6, the mutational profiles can be input into an encoder model. As discussed above, the encoder model can be trained to output a latent space representation (e.g., derived data) based on the plurality of mutational profiles. The encoder model can correspond to the encoder models described above, e.g., encoder models 120, 420.

With reference to FIG. 7, the encoder model 720 can receive a mutational profile 722 as an input. The encoder model 720 can be trained to compress the inputs via one or more neural network layers to a lower dimensional space and output respective latent space representations 726 of the mutation profiles 722. Compressing the data comprising the mutational profiles to the lower dimensional space forces the encoder model 720 to find consistent patterns that can be used to identify relationships (e.g., signatures) in the samples.

At block 608 of FIG. 6, the latent space representation can be input into a statistical model, e.g., an unsupervised machine learning model. In one or more examples, the unsupervised machine learning model can correspond to, but is not limited to, a density-based spatial clustering of applications with noise (DBSCAN), ordering points to identify the clustering structure (OPTICS); a gaussian mixture model (GMM), a k-means clustering model, a k nearest neighbors model, and/or a hierarchical clustering model. A skilled artisan will understand that other statistical models may be used at block 608 without departing from the scope of this disclosure.

With reference to FIG. 7, the statistical model 740 can receive the latent space representations 726 as an input. In one or more examples, the statistical model can group or cluster the latent space representations. Clustering the plurality of latent space representations of the mutational signatures can yield biologically relevant signatures as the clustering is based on one or more shared characteristics of the latent space representations.

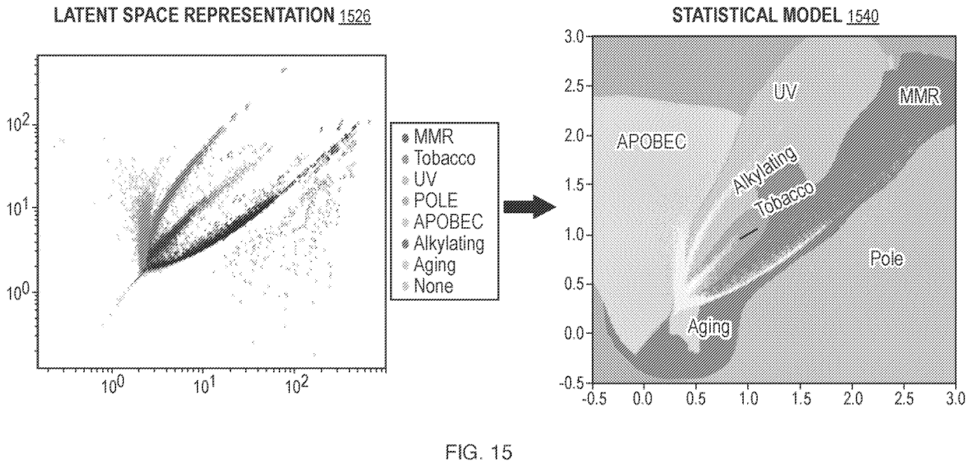

Referring briefly to FIG. 15, latent space representation 1526 illustrates exemplary graphical representation of a plurality of latent space representations. As shown in the figure, the latent space representations are clustered into regions, where a region can correspond to a mutational signature.

At block 610 of FIG. 6, the system can identify one or more mutational signatures (e.g., de novo mutational signatures) based on one or more clusters of latent space representations output by the statistical model. In some instances, one or more clusters may correspond to a de novo mutational signature. In some examples, the clustering methods may include, for example, but are not limited to k-means clustering, hierarchical clustering, density-based clustering, distribution-based clustering, similarity scoring, distance metrics, kernel target alignment (KTA) scoring, and the like. With reference to FIG. 7, the statistical model 740 can receive the latent space representations 726 as an input. The statistical model 740 can be configured to output one or more clusters, which can be used by the system to determine one or more mutational signatures 742.

### Autoencoder Training and Validation

Systems and methods of the present disclosure are further configured to train an autoencoder model comprising an encoder model (e.g., encoder model 120, 420, 720) and a decoder model (e.g., decoder model 130).

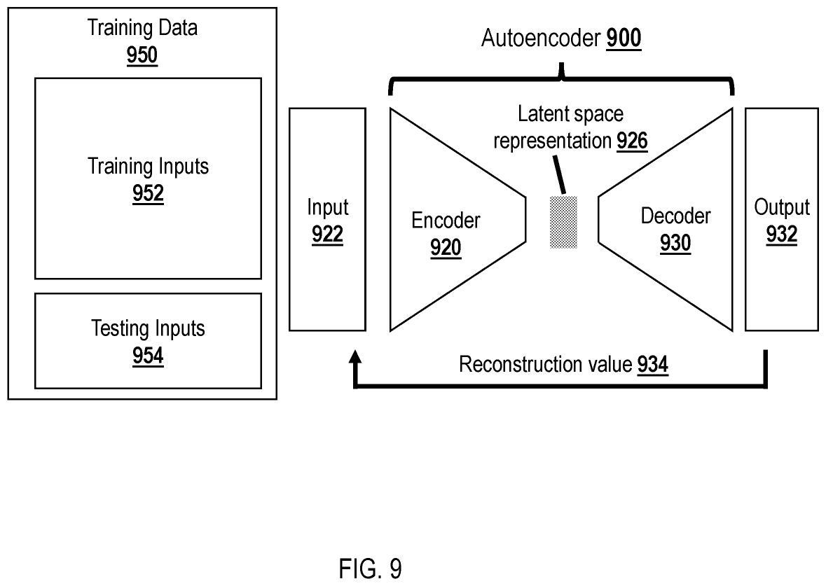

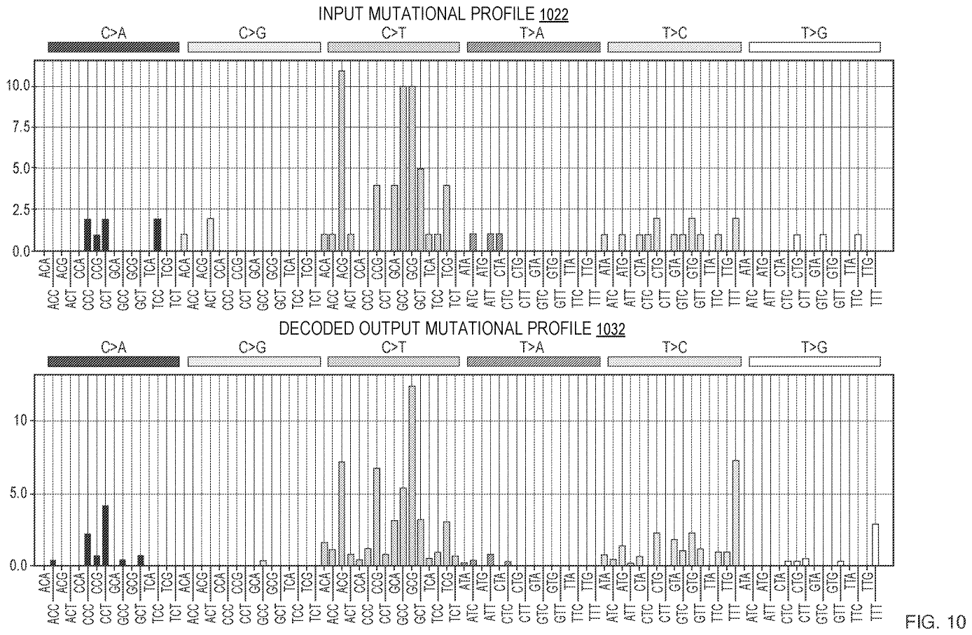

FIG. 8 illustrates an exemplary process 800 for training an autoencoder model in accordance with embodiments of the present disclosure. FIG. 9 illustrates an exemplary diagram of training an autoencoder model 900. Process 900 can be performed, for example, using one or more electronic devices implementing a software platform. In some examples, process 900 is performed using a client-server system, and the blocks of process 900 are divided up in any manner between the server and a client device. In other examples, the blocks of process 900 are divided up between the server and multiple client devices. Thus, while portions of process 900 are described herein as being performed by particular devices of a client-server system, it will be appreciated that process 900 is not so limited. In other examples, process 900 is performed using only a client device or only multiple client devices. In process 900, some blocks are, optionally, combined, the order of some blocks is, optionally, changed, and some blocks are, optionally, omitted. In some examples, additional steps may be performed in combination with the process 900. Accordingly, the operations as illustrated (and described in greater detail below) are exemplary by nature and, as such, should not be viewed as limiting.

At block 802 of FIG. 8, the system can receive training data comprising a plurality of mutational profiles from a plurality of training samples. For example, with reference to FIG. 9, the input 922 can correspond to training data 950. In some examples, the training data 950 can include training inputs 952 and testing inputs 954. In some examples, the training data 950 may be split such that 70% of the training data 950 comprises training inputs 952 used to train the model and 30% of the training data 950 comprises testing inputs 954 used to test the model. In some instances, the training data 950 may be split such that 80% of the training data 950 comprises training inputs 952 used to train the model and 20% of the training data comprises testing inputs 954. A skilled artisan will understand that other training inputs/test inputs splits may be used without departing from the scope of this disclosure.

In one or more examples, the training data may be obtained from one or more databases comprising sequence read data from a plurality of samples. In some instances, the system can process the sequence read data to obtain mutational profiles, as discussed above with respect to step 204 of FIG. 2 and step 604 of FIG. 6. In some instances, the mutational profiles may include single-base substitutions, double-base substitutions, insertions, deletions, and/or copy number alterations.

In some instances, the sequence read data corresponding to the training samples can be obtained from solid biopsy samples and/or liquid biopsy samples. In some instances, the sequence read data may be derived from single region sequencing, multi-region sequencing, single cell sequencing data (e.g., as opposed to bulk tumor sequencing). In some instances, the genomic data comprising sequence read data may be derived from sequencing the circulating tumor DNA in a liquid biopsy sample and/or RNA in a liquid biopsy sample.

In some instances, the genomic data comprising sequence read data may be derived from targeted sequencing, e.g., targeted exome sequencing, broad panel sequencing, whole genome, and/or whole exome sequencing. Unlike conventional techniques for identifying mutational signatures, embodiments of the present disclosure can be used with targeted exome sequencing, which allows mutational signatures having shorter lengths to input into the statistical model and provide robust results. In some examples, based on the type of sequencing used to derive the training data, the training data may correspond to a sequence length of less than 0.5 Megabase, 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, and/or greater than 2.0 Megabases. In one or more examples, the sequence read data may be received by the system as a BAM file.

In this manner, embodiments of the present disclosure can train a model to receive input data (e.g., mutational profiles) derived from targeted sequencing or panel sequencing that are relatively sparse compared to the mutational profiles derived from whole exome sequencing or whole genome sequencing. Because conventional methods to identify mutational signatures typically rely on mutational profiles derived from whole exome sequencing or whole genome sequencing, these conventional methods are unable to process samples with sparse data (e.g., mutational profiles derived from targeted or panel sequencing).

In some instances, the training data can be pre-processed to remove training samples. For example, conventional methods are unable to deduce patterns related to the mutational profiles for samples with a low number of mutations. However, embodiments in accordance with the present disclosure provide methods that allow the system to utilize samples that have a relatively low number of mutations compared to conventional methods for identifying mutational signatures. For example, conventional methods typically include 20 or more mutations or ten or more mutations. In some examples, the system can determine a number of mutations present in a training sample and if the number of mutations is less than a predetermined threshold, than the sample may be removed from the training data. In some examples, the predetermined threshold may be between three and twenty, e.g., the predetermined threshold may be 3, 5, 7, 10, 15, and/or 20 or more. Accordingly, embodiments of the present disclosure may use a wider range of samples compared to conventional methods.

At block 804 of FIG. 8, the system can input the plurality of mutational profiles into an untrained autoencoder model. For example, with reference to FIG. 9, the input 922 (e.g., comprising a plurality of mutational profiles) can be input into the encoder model 920 of the autoencoder model 900. In some examples, the encoder can include a plurality of layers (e.g., as shown in encoder model 120 of FIG. 1). In some examples, the encoder model 920 can be configured to output a latent space representation 926 of the input 922. For example, the encoder can be configured to compress the data corresponding plurality mutational signatures and output a latent space representation 926.

At block 806 of FIG. 8, the system can input the latent space representation into a decoder model of the untrained encoder model. For example, with reference to FIG. 9, the system can input the latent space representation 926 into the decoder model 930. At this stage in the process the decoder model 930 may be an untrained decoder model. The decoder model can be configured to reconstruct the mutational profiles of the input 922 based on the latent space representation 926 to produce an output 932.

At block 808 of FIG. 8, the system can determine a reconstruction value based on a comparison of the plurality of mutational profiles and the output of the decoder model. For example, with reference to FIG. 9, the system can compare the output 932 (e.g., corresponding to a reconstruction of the mutational profiles) of the decoder model 930 to the input 922 (e.g., corresponding to the plurality of mutational profiles) to determine a reconstruction value 934 (e.g., reconstruction error).

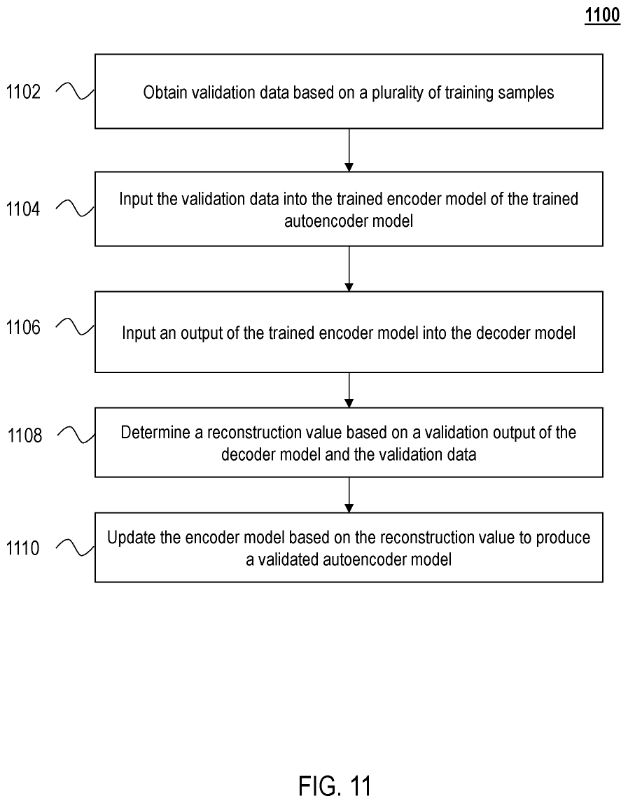

FIG. 10 illustrates an exemplary input mutational profile 1022 and an exemplary decoded output mutational profile 1032, in accordance with embodiments of the present disclosure. As shown in the figure, the decoded output mutational profile 1032 appears to be similar to the input mutational profile. Accordingly, the reconstruction value based on the input mutational profile 1022 and decoded output mutational profile 1032 would indicate these mutational profiles are similar. The reconstruction value can correspond to one or more numerical values representative of the differences between the input mutational profile 1022 and an exemplary decoded output mutational profile 1032.

At block 810 of FIG. 8, the system can update the encoder model based on the reconstruction value to produce a trained autoencoder model. For example, with reference to FIG. 9, the system can use the reconstruction value 934 to adjust one or more weights associated with the one or more layers of the encoder. In some instances, weights may be adjusted with respect to one or more dimensions of the latent space representation 926. In some instances, the system can similarly update one or more weights associated with the decoder model.

In some instances, a mutation signature autoencoder may be trained to process samples where the somatic and germline status cannot be called. In such instances, the training data may include all alterations (somatic and germline), e.g., such that the mutational profiles input 922 into the encoder model 920 for training include somatic and germline alterations. In such examples, instead of comparing the output 932 of the decoder model 930 to the input 922 (e.g., comprising the somatic and germline alterations) to determine the reconstruction value 934, the system may base the reconstruction value 934 based on the somatic alterations. In this manner, the model can be trained to ignore noise (e.g., germline alterations) when detecting signatures based on a sample.

In one or more examples, the system can validate the trained autoencoder model. In some instances, the validation can be used to determine whether the latent space representation of an input corresponding to sparse data, e.g., a mutational profile derived from targeted or panel sequencing, can be used to accurately reconstruct a full mutational profile of a sample. In one or more examples, the autoencoder model may be trained using sparse input data (e.g., mutational profiles derived from targeted or panel sequencing). The autoencoder can then be validated using validation data corresponding to non-sparse input data (e.g., mutational profiles derived from whole genome or whole exome sequencing).

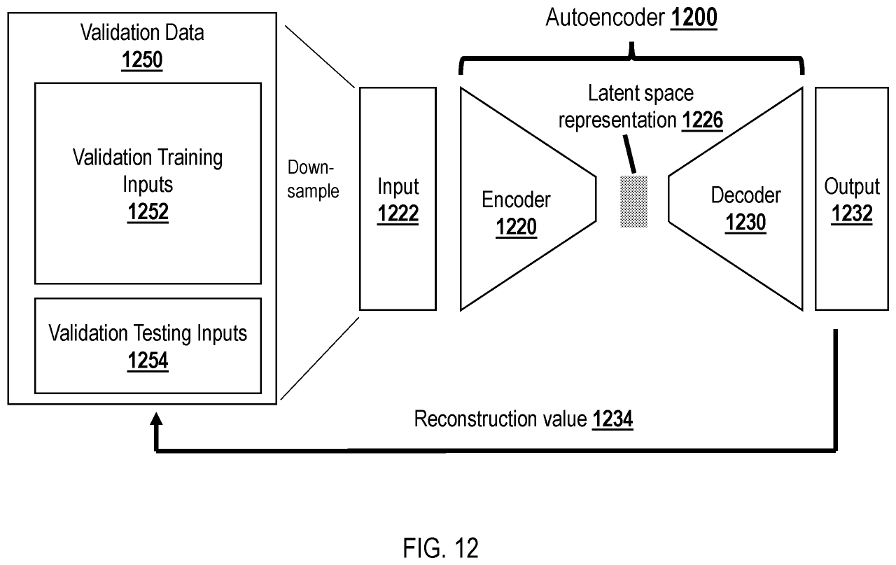

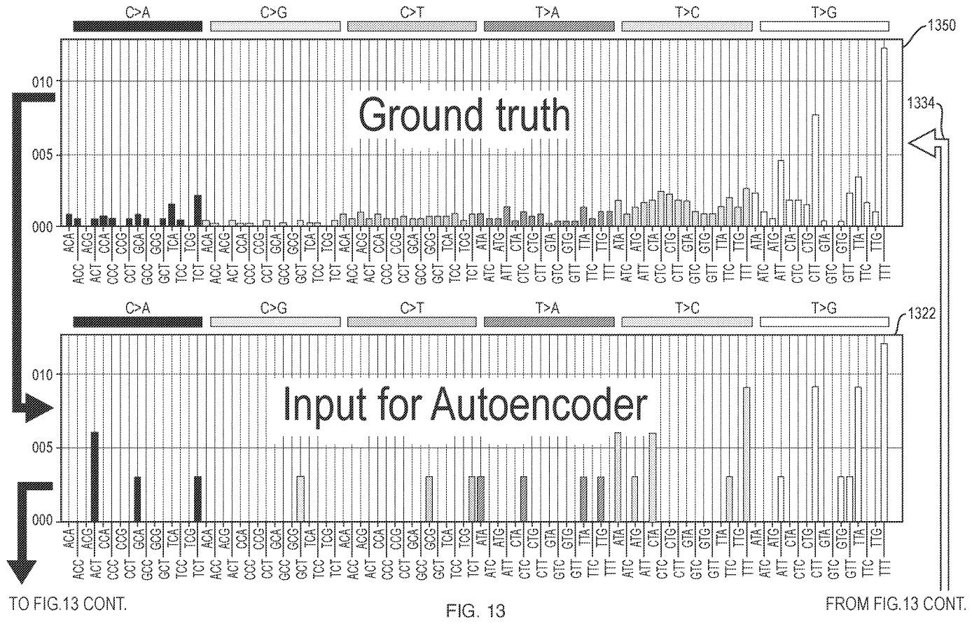

FIG. 11 illustrates an exemplary process 1100 for validating an autoencoder model in accordance with embodiments of the present disclosure. FIG. 12 illustrates an exemplary diagram of validating an autoencoder model 1200. Process 1100 can be performed, for example, using one or more electronic devices implementing a software platform. In some examples, process 1100 is performed using a client-server system, and the blocks of process 1100 are divided up in any manner between the server and a client device. In other examples, the blocks of process 1100 are divided up between the server and multiple client devices. Thus, while portions of process 1100 are described herein as being performed by particular devices of a client-server system, it will be appreciated that process 1100 is not so limited. In other examples, process 1100 is performed using only a client device or only multiple client devices. In process 1100, some blocks are, optionally, combined, the order of some blocks is, optionally, changed, and some blocks are, optionally, omitted. In some examples, additional steps may be performed in combination with the process 1100. Accordingly, the operations as illustrated (and described in greater detail below) are exemplary by nature and, as such, should not be viewed as limiting. In one or more examples, process 1100 corresponds to validating the latent space representation of a trained autoencoder model (e.g., latent space representation 926 of FIG. 9).

At block 1102 of FIG. 11, the system can obtain validation data comprising a plurality of mutational profiles from a plurality of validation samples. For example, with reference to FIG. 12, the validation data 1250 can include validation training inputs 1252 and validation testing inputs 1254. In one or more examples, the validation data 1250 may be a subset of training data 950. In some examples, the validation data 1250 may be split such that 70% of the validation data 1250 comprises validation training inputs 1252 used to train the model and 30% of the validation training data 1250 comprises testing inputs 1254 used to test the model. In some instances, the validation data 1250 may be split such that 80% of the validation data 1250 comprises validation training inputs 1252 used to train the model and 20% of the validation testing data comprises testing inputs 1254. A skilled artisan will understand that other training inputs/test inputs splits may be used without departing from the scope of this disclosure. In one or more examples, the validation training inputs 1252 and validation testing inputs 1254 correspond to different data sets.

In some embodiments, the validation data 1250 can be pre-processed to produce the input 1222 for the autoencoder 1200. For example, mutational profiles included in the validation data 1250 (e.g., mutational profiles) can be down-sampled to produce the input 1222.

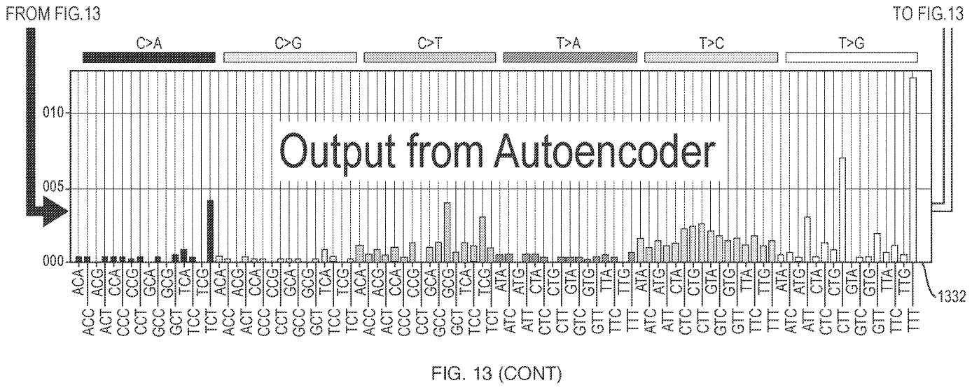

FIG. 13 illustrates exemplary validation data 1350 and exemplary input 1322. As shown in the figure, the validation data 1350 can correspond to a mutational profile. In some embodiments, the mutational profile may be derived based on whole genome or whole exome sequencing. The validation data 1350 can be down-sampled in order to produce input 1322. As shown in the figure, the down-sampled input 1322 lacks the specificity of the specific distribution of alterations identified in the validation data. In some examples, the down-sampled input 1322 can approximate the mutational profile obtained if the sample were processed using targeted or panel sequencing instead of whole genome or whole exome sequencing.

In one or more examples, the validation data 1250 may be obtained from one or more databases comprising sequence read data from a plurality of samples. In some instances, the system can process the sequence read data to obtain mutational profiles, as discussed above with respect to step 204 of FIG. 2 and step 604 of FIG. 6. In some instances, the mutational profiles may include single-base substitutions, double-base substitutions, insertions, deletions, and/or copy number alterations.

In some instances, the sequence read data corresponding to the validation samples can be obtained from solid biopsy samples and/or liquid biopsy samples. In some instances, the sequence read data may be derived from single region sequencing, multi-region sequencing, single cell sequencing data (e.g., as opposed to bulk tumor sequencing). In some instances, the genomic data comprising sequence read data may be derived from sequencing the circulating tumor DNA in a liquid biopsy sample and/or RNA in a liquid biopsy sample.

In some instances, the genomic data comprising sequence read data may be derived from targeted sequencing, e.g., targeted exome sequencing, broad panel sequencing, whole genome, and/or whole exome sequencing. In some examples, based on the type of sequencing used to derive the training data, the training data may correspond to a sequence length of less than 0.5 Megabase, 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, and/or greater than 2.0 Megabases. In one or more examples, the sequence read data may be received by the system as a BAM file.

At block 1104 of FIG. 11, the system can input the validation data into a trained encoder model of the trained autoencoder model. In some instances, the trained encoder model can be trained as described above with respect to process 800. In some instances, the validation data input into the trained encoder model can correspond to pre-processed validation data that has been down-sampled, e.g., input 1222, 1322. With reference to FIG. 12, the encoder 1220 can be configured to compress the input 1222 and output a latent space representation 1226.

At block 1106 of FIG. 11, the system can input the output of the encoder model into a decoder model. For example, with reference to FIG. 12, the latent space representation 1226 can be input into the decoder model 1230. As shown in the figure, the decoder model 1230 can be configured to reconstruct the mutational profiles of the validation data 1250 based on the latent space representation 1226. That is, instead of reconstructing the down-sampled input 1222, the decoder model 1230 can be trained to reconstruct the mutational profile of the original validation data 1250 as an output 1232.

An exemplary output 1332 from the autoencoder is illustrated in FIG. 13. The output from the autoencoder model may correspond to the output 1232 from the decoder model 1230. As shown in FIG. 13, the output 1332 from the autoencoder/decoder includes the specificity of the distribution of mutations similar to the validation data 1350.

At block 1108 of FIG. 11, the system can determine a reconstruction value based on the plurality of mutational profiles of the validation data and the output of the decoder model. For example, with reference to FIGS. 12 and 13, the reconstruction value 1234, 1334 can be determined based on the output of the decoder model 1232, 1332 and the validation data 1250, 1350 (e.g., instead of the input to the encoder model 1222, 1322).

At block 1110 of FIG. 11, the system can update the encoder model based on the reconstruction value to produce a validated autoencoder model. For example, with reference to FIG. 12, the system can use the reconstruction value 1234 to adjust one or more weights associated with the one or more layers of the encoder model 1220. In some instances, weights may be adjusted with respect to one or more dimensions of the latent space representation 1226. In some instances, the system can similarly update one or more weights associated with the decoder model 1230. In this manner, the validated autoencoder model can be configured to receive sparse data (e.g., a sparse mutational profile) as an input and reconstruct a full mutational profile based on the sparse data. In some examples, the encoder model may not be retrained based on the validation data. In some instances, the system can determine a concordance value between the validation data and the output of the validated decoder model.

In some instances, the trained and/or validated encoder model of the autoencoder model can be used to identify one or more mutational signatures in a sample as well as identify one or more de novo signatures associated with a plurality of samples. In some instances, the de novo signatures could be used as predictive and/or prognostic biomarkers.

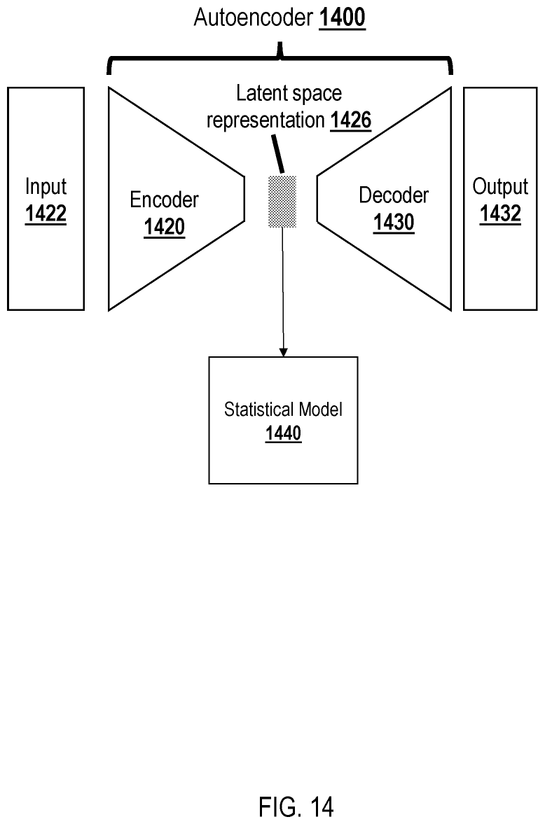

In one or more examples, the system can train a statistical model to identify one or more mutational signatures in a sample. FIG. 14 illustrates an exemplary block diagram for training a statistical model to identify one or more mutational signatures in accordance with embodiments of the present disclosure. As shown in the figure, the latent space representation 1426 of the autoencoder 1400 can be input into an untrained statistical model 1440. In one or more examples, autoencoder 1400 may correspond to autoencoder 900, once autoencoder 900 has been trained.

For example, the input 1422, may correspond to labeled mutational profile, where the labels correspond to one or more known mutational signatures associated with the sample. Based on these labels, the latent space representation 1426 can be used to train decision boundaries for model 1440, e.g., to determine whether a latent space representation of a mutational profile should be associated with a particular mutational signature.

FIG. 15 illustrates an exemplary graphical representation of a latent space representation 1526 corresponding to a plurality of samples in accordance with embodiments of this disclosure and how it can be used to train a statistical model to identify one or more mutational signatures. As shown in the figure, each dot may correspond to a mutational profile associated with a training sample. In some instances, each training sample may be associated with a known mutational signature. The latent space representation 1526 is color-coded such that each mutational profile associated with a known signature corresponds to a different color. As shown in the figure, samples associated with a particular mutational signature are clustered within a region in the graphical representation of the latent space 1526. Accordingly, the latent space representation 1526 may be used to train decision boundaries for the model 1540. As shown in the model 1540, the decision boundaries may define to regions in a latent space representation corresponding to a particular mutational signature, such that a mutational profile within a particular region is likely to correspond to the respective mutational profile, e.g., a mutational profile in the POLE region of the model 1540 is likely to correspond to the POLE signature being detected in a sample.

In some instances, the disclosed methods may be used to identify one or more mutational signatures by assessing the mutational profile associated with at least 1, at least 2, at least 3, at least 4, at least 5, at least 6, at least 7, at least 8, at least 9, at least 10, at least 20, at least 30, at least 40, or more than 40 gene loci.

In some instances, the disclosed methods may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (viii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for identifying mutational signatures in a sample from a subject may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for identifying mutational signatures in a sample from a subject may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for identifying mutational signatures in a sample from an individual may be used to select a subject (e.g., a patient) for a clinical trial based on the identified mutational signatures. In some instances, patient selection for clinical trials based on, e.g., identification of the mutational signatures, may accelerate the development of targeted therapies and improve the healthcare outcomes for treatment decisions.

In some instances, the disclosed methods for identifying mutational signatures in a sample from a subject may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), 53elinexor53b5353 sulfate (Vitrakvi), 53elinexor53 mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), 53elinexor53b53 (Gazyva), ofatumumab (Arzerra), 53elinexo (Lynparza), olaratumab (Lartruvo), 53elinexor53b (Tagrisso), 53elinexor53b (Ibrance), panitumumab (Vectibix), 53elinexor53b53 (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), 54elinexor54b govitecan-hziy (Trodelvy), seliciclib, 54elinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for identifying mutational signatures in a sample from a subject may be used in treating a disease (e.g., a cancer) in the subject. For example, in response to determining the mutational signatures using any of the methods disclosed herein, an effective amount of an anti-cancer therapy or anti-cancer treatment may be administered to the individual.

In some instances, the disclosed methods for identifying mutational signatures in a sample may be used for monitoring disease progression or recurrence (e.g., cancer or tumor progression or recurrence) in a subject. For example, in some instances, the methods may be used to determine the mutational signatures present in a first sample obtained from the subject at a first time point, and used to determine the mutational signatures present in a second sample obtained from the subject at a second time point, where comparison of the first determination of the mutational signatures of the first sample and the second determination of the mutational signatures of the second sample allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the mutational signatures identified in a sample from the subject.

In some instances, the mutational signatures determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for identifying mutational signatures in a sample from a subject may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for identifying mutational signatures in a sample from a subject as part of a genomic profiling process (or inclusion of the output from the disclosed methods for identifying mutational signatures as part of the genomic profile of the subject) can improve the validity of, e.g., disease detection calls and treatment decisions, made on the basis of the genomic profile by, for example, independently confirming the presence of mutational signatures in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17 p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AmoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM 333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB 351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an Rnase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM 1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T.J. et al. (2007) Nat. Methods 4(11):903-5; Hodges, E. et al. (2007) Nat. Genet. 39(12):1522-7; and Okou, D.T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; I aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S.L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D.R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertio-s—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981“, “Identification of Common Molecular Subsequen”es”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (197“) “A General Method Applicable to the Search for Similarities in the Amino Acid Sequence of Two Prote”ns”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loc.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite-or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5 mC and 5 hmC. The method combines ten-eleven translocation methylcytosine dioxygenase (TET)-mediated oxidation of 5 mC and 5 hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11):1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B.L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e−6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C.A., et al., Genome Res. 2011;21(6):961-73). For example, the Bayesian EM algorithm can be used to analyze the reads, make initial indel calls, and generate genotype likelihoods for each candidate indel, followed by imputation of genotypes using, e.g., QCALL (Le S.Q. and Durbin R. Genome Res. 2011;21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequenci-g—A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski JK, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for identifying mutational signatures in a sample from a subject. The systems may comprise, e.g., one or more processors, and a memory unit communicatively coupled to the one or more processors and configured to store instructions that, when executed by the one or more processors, cause the system to: receive sequence read data associated with the sample; determine a mutational profile of the sample based on the sequence read data; input the mutational profile into an encoder model, wherein the encoder model is trained using training data related to a plurality of mutational signatures; and predict one or more mutational signatures of the plurality of mutational signatures associated with the sample based on an output of the encoder model, wherein the output of the encoder model is associated with a dimensionality value, and wherein the dimensionality value is less than a number of the plurality of mutational signatures.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for identifying mutational signatures in any of a variety of samples as described herein (e.g., a tissue sample, biopsy sample, hematological sample, or liquid biopsy sample derived from the subject).

In some instances, the plurality of gene loci for which sequencing data is processed to identify mutational signatures may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of mutational signatures in a sample is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

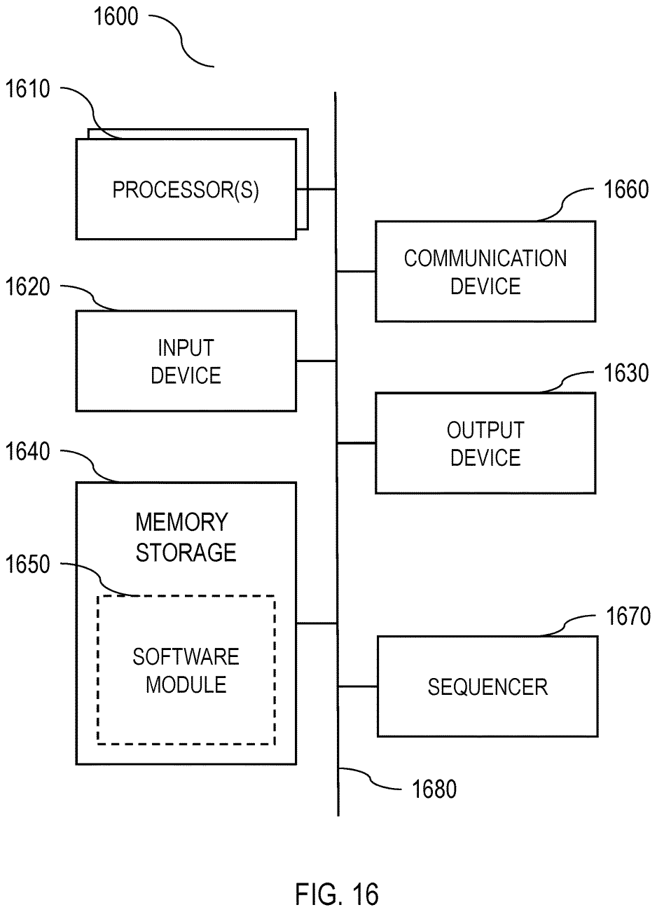

FIG. 16 illustrates an example of a computing device or system in accordance with one embodiment. Device 1600 can be a host computer connected to a network. Device 1600 can be a client computer or a server. As shown in FIG. 16, device 1600 can be any suitable type of microprocessor-based device, such as a personal computer, workstation, server or handheld computing device (portable electronic device) such as a phone or tablet. The device can include, for example, one or more processor(s) 1610, input devices 1620, output devices 1630, memory or storage devices 1640, communication devices 1660, and nucleic acid sequencers 1670. Software 1650 residing in memory or storage device 1640 may comprise, e.g., an operating system as well as software for executing the methods described herein. Input device 1620 and output device 1630 can generally correspond to those described herein, and can either be connectable or integrated with the computer.

Input device 1620 can be any suitable device that provides input, such as a touch screen, keyboard or keypad, mouse, or voice-recognition device. Output device 1630 can be any suitable device that provides output, such as a touch screen, haptics device, or speaker.

Storage 1640 can be any suitable device that provides storage (e.g., an electrical, magnetic or optical memory including a RAM (volatile and non-volatile), cache, hard drive, or removable storage disk). Communication device 1660 can include any suitable device capable of transmitting and receiving signals over a network, such as a network interface chip or device. The components of the computer can be connected in any suitable manner, such as via a wired media (e.g., a physical system bus 1680, Ethernet connection, or any other wire transfer technology) or wirelessly (e.g., Bluetooth®, Wi-Fi®, or any other wireless technology).

Software module 1650, which can be stored as executable instructions in storage 1640 and executed by processor(s) 1610, can include, for example, an operating system and/or the processes that embody the functionality of the methods of the present disclosure (e.g., as embodied in the devices as described herein).

Software module 1650 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1640, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 1650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

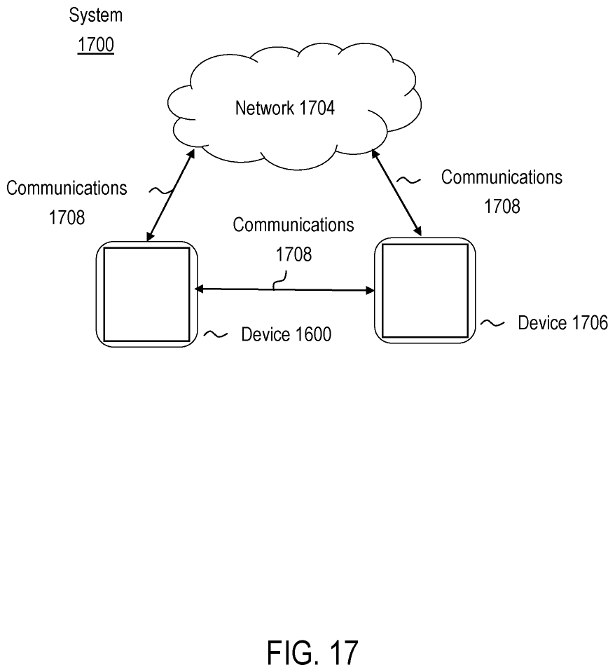

Device 1600 may be connected to a network (e.g., network 1704, as shown in FIG. 17 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1600 can be implemented using any operating system, e.g., an operating system suitable for operating on the network. Software module 1650 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 1610.

Device 1600 can further include a sequencer 1670, which can be any suitable nucleic acid sequencing instrument.

FIG. 17 illustrates an example of a computing system in accordance with one embodiment. In system 1700, device 1600 (e.g., as described above and illustrated in FIG. 16) is connected to network 1704, which is also connected to device 1706. In some embodiments, device 1706 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 1600 and 1706 may communicate, e.g., using suitable communication interfaces via network 1704, such as a Local Area Network (LAN), Virtual Private Network (VPN), or the Internet. In some embodiments, network 1704 can be, for example, the Internet, an intranet, a virtual private network, a cloud network, a wired network, or a wireless network. Devices 1600 and 1706 may communicate, in part or in whole, via wireless or hardwired communications, such as Ethernet, IEEE 802.11b wireless, or the like. Additionally, devices 1600 and 1706 may communicate, e.g., using suitable communication interfaces, via a second network, such as a mobile/cellular network. Communication between devices 1600 and 1706 may further include or communicate with various servers such as a mail server, mobile server, media server, telephone server, and the like. In some embodiments, Devices 1600 and 1706 can communicate directly (instead of, or in addition to, communicating via network 1704), e.g., via wireless or hardwired communications, such as Ethernet, IEEE 802.11b wireless, or the like. In some embodiments, devices 1600 and 1706 communicate via communications 1708, which can be a direct connection or can occur via a network (e.g., network 1704).

One or all of devices 1600 and 1706 generally include logic (e.g., http web server logic) or are programmed to format data, accessed from local or remote databases or other sources of data and content, for providing and/or receiving information via network 1704 according to various examples described herein.

### EXAMPLES

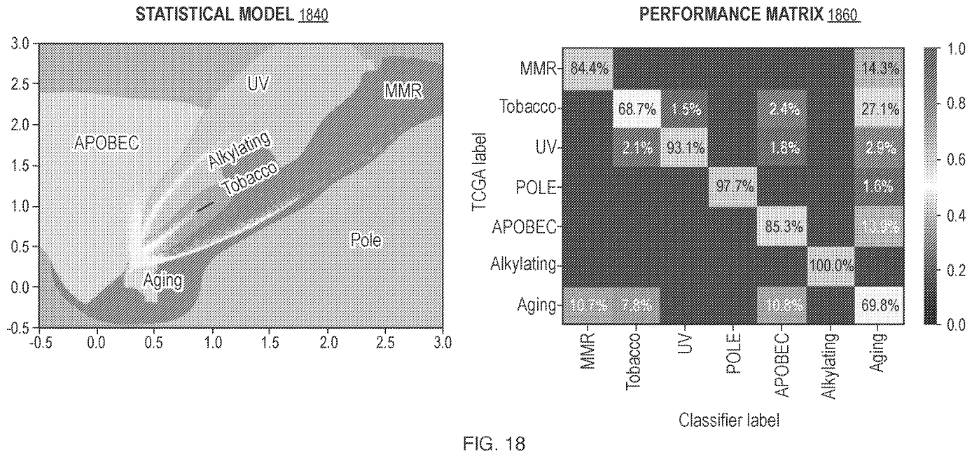

FIG. 18 illustrates a non-limiting example of a statistical model 1840 trained to identify one or more mutational signatures in accordance with embodiments of the present disclosure. As shown in the figure, the statistical model 1840 can include one or more regions associated with various mutational signatures. The statistical model 1840 can be trained to receive latent space representations (e.g., the latent space representations output by an encoder model as discussed above). The white dots correspond to various latent space representations input into the statistical model. FIG. 18 also includes performance metrics corresponding to the accuracy of the model. As shown in the figure, the values along the diagonal represent the number of correct calls produced by the statistical model 1840. As shown in performance metrics 1860, the statistical model has an accuracy of above 90% for the MMR, Tobacco, UV, POLE, APOBEC, Alkylating Agent Resistance, and Aging signatures. Accordingly, embodiments of the present disclosure can be used to accurately predict one or more mutational signatures.

Embodiments of the present disclosure can be used to identify, based on patient samples, one or more alkylating agent resistance profiles that are indicative of the patient's resistance to alkylating agents. Alkylating agents are a diverse class of cytotoxic chemotherapeutic drugs that inhibit cell division by damaging DNA. These therapies are used to treat a wide variety of neoplasms, including those of the brain, lung, breast, and ovary as well as leukemia, lymphoma, Hodgkin disease, multiple myeloma, and sarcoma. However, many patients eventually develop resistance to alkylating agents. In current clinical practice, detection of resistance is tied to measures of poor prognosis and worsening disease, including methylguanine methyltransferase (MGMT) promoter methylation testing, growth of tumor on imaging, changes in blood-based biomarkers, or exacerbated symptomology for the patient. These paradigms identify resistance when the cancer disease burden has already increased. However, there is often a lag between the reemergence of cancer and the detection of alkylating resistance in a patient based on routine scans and other biomarkers (e.g., blood-based biomarkers such as, but not limited to, PSA for prostate cancer).

Compared to conventional methods of determining resistance to alkylating agents, embodiments of the present disclosure can provide earlier detection of resistance to alkylating agents based on an alkylating agent resistance signature profile determined via blood or tissue assays. Earlier detection can lead to earlier changes in therapy and improved outcomes. Accordingly, embodiments of the present disclosure aim to leverage an alkylating agent resistance signature as a sensitive and specific biomarker for emerging resistance. In one or more examples, the alkylating agent resistance signature may correspond to the COSMIC signature 11.

Like other cancer therapeutics, the mechanism for resistance to alkylating agents is a product of its mechanism of action. The DNA damaging effect is mediated in a highly chemically specific manner wherein an alkyl group is attached to th^{e} 7th nitrogen atom of the purine ring of guanine bases. Resistance to alkylating agents is mediated by the DNA repair enzyme O6-methylguanine methyltransferase (MGMT), which excises the adduct prior to replication and thereby eliminating the cytotoxic effect. Due to this specific interaction between the mechanisms of drug action and resistance, the cancer genome of resistant patients can include characteristic patterns of mutations. For example, patients with alkylating resistance may have C>T mutations in a characteristic CC/CT dinucleotide context. Embodiments of the present disclosure can quantify this pattern to build an alkylating agent resistance profile (e.g., alkylating mutational profile) for a patient that can be tracked over time and used as a biomarker for resistance to alkylating agents.

The alkylating agent resistance profile of a patient may be compared to a pre-determined alkylating agent resistance signature to determine if a patient is resistant to treatment with alkylating agents. In one or more examples, the alkylating agent resistance mutational signature may correspond to a trinucleotide context-specific C>T substitution pattern. In some examples, this substitution pattern may occur in a NCC or a NCT trinucleotide context (e.g., where N corresponds to any of the four bases).

Methods according to examples of this disclosure can be used to determine whether a patient has developed resistance to an alkylating agent. For example the method can include receiving, using one or more processors, sequence read data associated with a sample from the patient, selecting, using the one or more processors, a plurality of reads from the sequence read data, determining, using the one or more processors, an alkylating agent resistance mutational profile based on the selected plurality of reads, inputting, using the one or more processors, the alkylating mutational profile into a statistical model (e.g., an encoder model as described above), and predicting, using the one or more processors, an alkylating resistance of the patient based on an output of the statistical model.

In one or more examples the output of the statistical model may be indicative of the alkylating agent resistance signature status. In one or more examples, the output of the statistical model may be indicative of a fit of the alkylating agent resistance profile to the alkylating agent resistance signature. In one or more examples, if the alkylating agent resistance profile is indicative of a positive status (e.g., the profile is indicative of the presence of an alkylating agent resistance signature), the patient may be determined to be resistant to the alkylating agent. In response to a positive status, a healthcare provider may modify the treatment and/or therapy of the patient. For example, if the patient was previously being treated with an alkylating agent, the healthcare provider may switch the treatment to a non-alkylating agent therapy.

In one or more examples, the output of the statistical model may further be processed by a second statistical model. For example, the output of the statistical model (e.g., encoder model) may be input into the second statistical model along with additional features, such as, but not limited to a imaging results, blood test results, genomic data, demographic data, and the like. In such examples, the output of this second model may be indicative of the alkylating agent resistance signature status. As in the example described above, if the patient is determined to have a positive status for the alkylating agent resistance signature, a healthcare provider may modify the treatment and/or therapy of the patient to no longer treat the patient with an alkylating agent.

In one or more examples, determining the alkylating agent resistance profile of a sample may be based on a variety of alterations, e.g., as discussed above, including, but not limited to single nucleotide substitution, multi-nucleotide substitution, insertions/deletions, copy number alterations, mutations in RNA, quantitative gene expression profiling, and/or whole exome sequencing. In one or more examples, the samples may be collected at a single time point or collected regularly as a part of ongoing treatment of the patient.

Embodiments of the present disclosure can be used to examine patients. For example, in a first analysis, embodiments of the present disclosure were used with data associated with longitudinal profiling based on tissue or liquid biopsy-based assays (N=7,079) obtained from one or more databases. In this example, 45 cases showed an acquisition of an alkylating agent resistance signature in the second biopsy. These cases were predominantly gliomas (N=36), likely associated with temolozomide (TMZ) standard of care treatment in these tumors. Additional tumor types with an acquired alkylating agent resistance signature included neuroendocrine tumors, colorectal cancer (CRC), and non-small cell lung cancer (NSCLC), among others. Further investigation into these additional tumors showed a median tumor mutational burden (TMB) increase of 87 mutations/Mb [IQR 34-115 mutations/Mb]. Based on this analysis, the acquisition of an alkylating agent resistance profile indicative of alkylating agent resistance was found to occur across a wide range of time between biopsies, which may include intervening therapies (0-11 years).

In a second analysis, embodiments of the present disclosure were used to examine data from patients corresponding to liquid biopsies associated with a second database. Of the patient data included in the second database, 22,183 patients were determined to have evaluable mutational signatures (e.g., circulating tumor fraction greater than or equal to 1% and an overall passing quality control status). Of these patients, eighteen cases were found to include an alkylating agent resistance signature. These eighteen cases spanned several tumor types including breast, CRC, prostate, non-small cell lung cancer (NSCLC), and neuroendocrine tumors, among others. These patients presented with a median blood-based TMB (bTMB) of 12 mutations/Mb [IQR 9-112 mutations/Mb]. Eleven of the eighteen cases had a bTMB greater than or equal to 10 mutations/Mb. This analysis suggests resistance/progression on treatment and may benefit from re-evaluation/switching of therapy.

Patients who progress due to alkylating agent resistance with an associated alkylating agent resistance signature may harbor many alterations, some of which are recurrent and potentially actionable such that an alkylating agent resistance signature can indicate alternative methods of treatment. For example, in order to analyze an alkylating agent resistance signature as a potential indicator of resistance to TMZ treatment, a second analysis interrogated gene alterations in 231 cases with an alkylating agent resistance signature (e.g., alkylating agent resistance positive status) and 8,515 samples that lacked an alkylating agent resistance signature (e.g., alkylating agent resistance non-positive status). Based on this analysis, 98% of the alkylating agent resistance positive cases also included a high tumor mutational burden (TMB) (e.g., a tumor mutational burden greater than or equal to10 mutations/Mb). In the alkylating agent resistance non-positive cohort 2% of the cases included a high TMB. This analysis suggests the alkylating agent resistance signature can predict an increase in TMB, which may, in turn, predict a patient's response to immune checkpoint inhibitors. Additionally, based on the analysis alterations in MSH6, MSH2 and MLH1 were determined to be enriched in alkylating agent resistance positive status brain tumors, further suggesting immunotherapy options for these cases. Additionally, alterations in PIK3CA, TP53, among others, were also detected more frequently in alkylating agent resistance positive status brain tumors. These data suggest the enrichment of targetable alterations (especially those with FDA approved drugs) in alkylating agent resistance positive status brain tumors relative to alkylating agent resistance non-positive status brain tumors. Accordingly, this analysis suggests earlier detection of resistance to alkylating agents via blood or tissue assays can allow healthcare providers to select changes in therapy to improve outcomes for patients.

### EXEMPLARY IMPLEMENTATIONS

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads;
   determining, using the one or more processors, a mutational profile of the sample based on the sequence read data;
   inputting, using the one or more processors, the mutational profile into an autoencoder model, wherein the autoencoder model is trained using training data related to a plurality of mutational signatures; and
   predicting, using the one or more processors, one or more mutational signatures of the plurality of mutational signatures associated with the sample based on an output of the autoencoder model,
   wherein the output of the autoencoder model is associated with a dimensionality value, and wherein the dimensionality value is less than a number of the plurality of mutational signatures.
2. The method of clause 1, wherein the autoencoder model comprises multiple layers.
3. The method of any of clauses 1 to 2, wherein the plurality of mutational signatures corresponds to a plurality of predefined mutational signatures.
4. The method of any of clauses 1 to 3, wherein the plurality of mutational signatures corresponds to a plurality of COSMIC mutational signatures.
5. The method of any of clauses 1 to 4, wherein the plurality of predefined mutational signatures comprise a DNA mismatch repair (MMR) signature, a tobacco signature, a UV signature, a putative polymerase epsilon (POLE) signature, an apolipoprotein B mRNA-editing enzyme, catalytic polypeptide (APOBEC) signature, an alkylating agent resistance signature, or a combination thereof.
6. The method of any of clauses 1 to 5, wherein predicting the one or more mutational signatures comprises inputting the output of the autoencoder model into a statistical model, the statistical model comprising at least one of a random forest model, a gradient boosting model, a logistic regression model, a support vector machine (SVM), a decision tree model, a density-based spatial clustering of applications with noise (DBSCAN), ordering points to identify the clustering structure (OPTICS); a gaussian mixture model (GMM), a k-means clustering model, a k nearest neighbors model, or a hierarchical clustering model.
7. The method of clause 6, further comprising training the statistical model, wherein the statistical model comprises a classifier model, wherein training the classifier model comprises:
   inputting, using the one or more processors, a plurality of training mutational profiles into the autoencoder model;
   inputting, using the one or more processors, outputs of the autoencoder model corresponding to the plurality of training mutational profiles into an unlabeled classifier model; and
   identifying, using the one or more processors, one or more mutational signatures based on clustering of the outputs of the autoencoder model.
8. The method of any of clauses 1 to 7, wherein the mutational profile comprises single-base substitutions.
9. The method of any one of clauses 1 to 8, wherein the subject is suspected of having or is determined to have cancer.
10. The method of clause 9, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ew'ng's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wi'ms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
11. The method of clause 9, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17 p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castle'an's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenst'om's macroglobulinemia.
12. The method of clause 11, further comprising treating the subject with an anti-cancer therapy.
13. The method of clause 12, wherein the anti-cancer therapy comprises a targeted anti-cancer therapy.
14. The method of clause 13, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb99elinexor99b9999nib sulfate (Vitrakvi99elinexor99nib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo99elinexor99b99mab (Gazyva), ofatumumab (Arzerra99elinexorib (Lynparza), olaratumab (Lartruvo99elinexor99bnib (Tagrisso99elinexor99blib (Ibrance), panitumumab (Vectibix99elinexor99b99tat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi99elinexor99bmab govitecan-hziy (Trodelvy), selicicli99elinexorxor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
15. The method of any one of clauses 1 to 14, further comprising obtaining the sample from the subject.
16. The method of any one of clauses 1 to 15, wherein the sample comprises a tissue biopsy sample, a liquid biopsy sample, or a normal control.
17. The method of clause 16, wherein the sample is a liquid biopsy sample and comprises blood, plasma, cerebrospinal fluid, sputum, stool, urine, or saliva.
18. The method of clause 16, wherein the sample is a liquid biopsy sample and comprises circulating tumor cells (CTCs).
19. The method of clause 16, wherein the sample is a liquid biopsy sample and comprises cell-free DNA (cfDNA), circulating tumor DNA (ctDNA), or any combination thereof.
20. The method of any one of clauses 1 to 19, wherein the plurality of nucleic acid molecules comprises a mixture of tumor nucleic acid molecules and non-tumor nucleic acid molecules.
21. The method of clause 20, wherein the tumor nucleic acid molecules are derived from a tumor portion of a heterogeneous tissue biopsy sample, and the non-tumor nucleic acid molecules are derived from a normal portion of the heterogeneous tissue biopsy sample.
22. The method of clause 20, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
23. The method of any one of clauses 1 to 22, wherein the one or more adapters comprise amplification primers, flow cell adaptor sequences, substrate adapter sequences, or sample index sequences.
24. The method of any one of clauses 1 to 23, wherein the captured nucleic acid molecules are captured from the amplified nucleic acid molecules by hybridization to one or more bait molecules.
25. The method of clause 24, wherein the one or more bait molecules comprise one or more nucleic acid molecules, each comprising a region that is complementary to a region of a captured nucleic acid molecule.
26. The method of any one of clauses 1 to 25, wherein amplifying nucleic acid molecules comprises performing a polymerase chain reaction (PCR) amplification technique, a non-PCR amplification technique, or an isothermal amplification technique.
27. The method of any one of clauses 1 to 26, wherein the sequencing comprises use of a massively parallel sequencing (MPS) technique, whole genome sequencing (WGS), whole exome sequencing, targeted sequencing, direct sequencing, or Sanger sequencing technique.
28. The method of clause 27, wherein the sequencing comprises massively parallel sequencing, and the massively parallel sequencing technique comprises next generation sequencing (NGS).
29. The method of any one of clauses 1 to 28, wherein the sequencer comprises a next generation sequencer.
30. The method of any one of clauses 1 to 29, wherein one or more of the plurality of sequencing reads overlap one or more gene loci within one or more subgenomic intervals in the sample.
31. The method of clause 30, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
32. The method of clause 30 or clause 31, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4(C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
33. The method of clause 30 or clause 31, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.
34. The method of any one of clauses 1 to 33, further comprising generating, by the one or more processors, a report indicating the one or more predicted mutational signatures.
35. The method of clause 34, further comprising transmitting the report to a healthcare provider.
36. The method of clause 35, wherein the report is transmitted via a computer network or a peer-to-peer connection.
37. A method for identifying mutational signatures associated with a sample from an individual, the method comprising:
   receiving, using one or more processors, sequence read data associated with the sample;
   determining, using the one or more processors, a mutational profile of the sample based on the sequence read data;
   inputting, using the one or more processors, the mutational profile into an encoder model, wherein the encoder model is trained using training data related to a plurality of mutational signatures; and
   predicting, using the one or more processors, one or more mutational signatures of the plurality of mutational signatures associated with the sample based on an output of the encoder model,
   wherein the output of the encoder model is associated with a dimensionality value, and wherein the dimensionality value is less than a number of the plurality of mutational signatures.
38. The method of clause 37, wherein the encoder model comprises multiple layers.
39. The method of any of clauses 37 to 38, wherein the plurality of mutational signatures corresponds to a plurality of predefined mutational signatures.
40. The method of any of clauses 37 to 39, wherein the plurality of mutational signatures corresponds to a plurality of COSMIC mutational signatures.
41. The method of any of clauses 37 to 40, wherein the plurality of predefined mutational signatures comprise a DNA mismatch repair (MMR) signature, a tobacco signature, a UV signature, a putative polymerase epsilon (POLE) signature, an apolipoprotein B mRNA-editing enzyme, catalytic polypeptide (APOBEC) signature, an alkylating agent resistance signature, or a combination thereof.
42. The method of any of clauses 37 to 41, wherein predicting the one or more mutational signatures comprises inputting the output of the encoder model into a statistical model.
43. The method of clause 42, wherein the statistical model comprises at least one of a random forest model, a gradient boosting model, a logistic regression model, a support vector machine (SVM), a decision tree model, a density-based spatial clustering of applications with noise (DBSCAN), ordering points to identify the clustering structure (OPTICS); a gaussian mixture model (GMM), a k-means clustering model, a k nearest neighbors model, or a hierarchical clustering model.
44. The method of clause 42, further comprising training the statistical model, wherein the statistical model is a classifier model, wherein training the statistical model comprises:
   inputting, using the one or more processors, a plurality of training mutational profiles into the encoder model;
   inputting, using the one or more processors, outputs of the encoder model corresponding to the plurality of training mutational profiles into an unlabeled classifier model; and
   identifying, using the one or more processors, one or more mutational signatures based on clustering of the outputs of the encoder model.
45. The method of any of clauses 37 to 44, wherein the mutational profile comprises less than twenty mutations.
46. The method of clause 45, wherein the mutational profile comprises at least three mutations.
47. The method of any of clauses 37 to 46, wherein the mutational profile is based on a targeted-sequencing panel or a targeted-exome sequencing panel.
48. The method of any of clauses 37 to 46, wherein the mutational profile is based on a whole exome sequencing technique or a whole genome sequencing technique.
49. The method any of clauses 37 to 48, wherein the sequence read data associated with the sample is derived from a single biopsy sample.
50. The method any of clauses 37 to 48, wherein the sequence read data associated with the sample is derived from multiple biopsy samples.
51. The method any of clauses 37 to 50, wherein the sequence read data associated with the sample is derived from single cell sequencing.
52. The method any of clauses 37 to 51, wherein the sequence read data associated with the sample is derived from circulating tumor DNA in a liquid biopsy sample.
53. The method any of clauses 37 to 52, wherein the sequence read data associated with the sample is derived from RNA in a liquid biopsy sample.
54. The method of any of clauses 37 to 53, wherein the mutational profile comprises single-base substitutions.
55. The method of any of clauses 37 to 54, wherein the mutational profile comprises double-base substitutions, insertions, deletions, copy number, rearrangement information, or a combination thereof.
56. The method of any of clauses 37 to 55, wherein the training data comprises training mutational profiles comprising single-base substitutions, double-base substitutions, insertions, deletions, copy number, rearrangement information, or a combination thereof.
57. The method of any of clauses 37 to 56, wherein the encoder model is trained by:
   receiving, using the one or more processors, the training data based on a plurality of training samples; and
   training, using the one or more processors, an autoencoder model comprising the encoder model based on the training data to obtain a trained autoencoder model.
58. The method of clause 57, wherein training the autoencoder model comprises:
   inputting, using the one or more processors, the training data into an untrained encoder model;
   inputting, using the one or more processors, an output of the untrained encoder model into a decoder model of the autoencoder model;
   determining, using the one or more processors, a reconstruction value of an output of the decoder model based on the training data and the output of the decoder model; and
   updating, using the one or more processors, the untrained encoder model based on the reconstruction value.
59. The method of any of clauses 57 to 58, wherein the training data corresponds to a sequence length of 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, or a combination thereof.
60. The method of any of clauses 57 to 58, wherein the training data corresponds to a sequence length less than 0.5 Megabases.
61. The method of any of clauses 57 to 58, wherein the training data corresponds to a sequence length greater than 2.0 Megabases.
62. The method of any of clauses 58 to 61, wherein the training data comprises germline alterations and somatic alterations and the reconstruction value is based on the somatic alterations.
63. The method of any of clauses 57 to 62, further comprising:
   determining a number of mutations present in a training sample of the plurality of training samples;
   removing the training sample from the training data if the number of mutations present in the training sample is below a predetermined threshold.
64. The method of clause 63, wherein the predetermined threshold is greater than three.
65. The method of any of clauses 57 to 64, further comprising validating the trained autoencoder model, the validating comprising:
   obtaining, using the one or more processors, validation data based on the plurality of training samples, wherein the training data comprises the validation data; and
   inputting, using the one or more processors, the validation data into a trained encoder model of the trained autoencoder model;
   inputting, using the one or more processors, an output of the trained encoder model into the decoder model;
   determining, using the one or more processors, a reconstruction value of a validation output of the decoder model based on the validation data; and
   updating, using the one or more processors, the encoder model based on the reconstruction value.
66. The method of any of clauses 57 to 64, further comprising validating the trained autoencoder model, the validating comprising:
   obtaining, using the one or more processors, validation data based on the plurality of training samples, wherein the training data comprises the validation data; and
   inputting, using the one or more processors, the validation data into a trained encoder model of the trained autoencoder model;
   inputting, using the one or more processors, an output of the trained encoder model into the decoder model;
   determining, using the one or more processors, a concordance value between the training data and a validation output of the decoder model; and
   updating, using the one or more processors, the encoder model based on the concordance value.
67. The method of any one of clauses 65 to 66, wherein obtaining the validation data comprises:
   receiving, by the one or more processors, raw validation data;
   down sampling, using the one or more processors, the raw validation data to obtain the validation data, and
   wherein the reconstruction value or the concordance value is determined based on the raw validation data.
68. The method of clause 67, wherein determining the reconstruction value is based on the somatic alterations comprising the raw validation data and the somatic alterations comprising the validation data.
69. The method of any of clauses 64 to 68, wherein the validation data is associated with sequence lengths of 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, or a combination thereof.
70. The method of any of clauses 64 to 68, wherein the training data corresponds to a sequence length less than 0.5 Megabases.
71. The method of any of clauses 64 to 68, wherein the training data corresponds to a sequence length greater than 2.0 Megabases.
72. The method any of clauses 37 to 71, further comprising assigning, using the one or more processors, a therapy for the individual based on the one or more mutational signatures.
73. The method any of clauses 37 to 72, further comprising administering, using the one or more processors, a treatment to the individual based on the one or more mutational signatures.
74. The method any of clauses 37 to 73, further comprising associating, using the one or more processors, the individual with a clinical trial based on the one or more mutational signatures.
75. The method any of clauses 37 to 74, further comprising monitoring, using the one or more processors, a prognosis of the individual based on the one or more mutational signatures.
76. The method any of clauses 37 to 75, further comprising predicting, using the one or more processors, one or more clinical outcomes based on the one or more mutational signatures.
77. A method for diagnosing a disease, the method comprising: diagnosing that an individual has a disease based on a determination of a mutational signature associated with a sample from the individual, wherein the mutational signature is determined according to the method of any one of clauses 37 to 76.
78. A method of selecting an anti-cancer therapy, the method comprising: responsive to determining a mutational signature associated with sample from an individual, selecting an anti-cancer therapy for the individual, wherein mutational signature is determined according to the method of any one of clauses 37 to 76.
79. A method of treating a cancer in a individual, comprising: responsive to determining a mutational signature associated with a sample from an individual, administering an effective amount of an anti-cancer therapy to the individual, wherein the mutational signature is determined according to the method of any one of clauses 37 to 76.
80. A method for monitoring cancer progression or recurrence in a individual, the method comprising:
   determining a first mutational signature in a first sample obtained from the individual at a first time point according to the method of any one of clauses 37 to 76;
   determining a second mutational signature in a second sample obtained from the individual at a second time point; and
   comparing the first mutational signature to the second mutational signature, thereby monitoring the cancer progression or recurrence.
81. The method of clause 80, wherein the second mutational signature for the second sample is determined according to the method of any one of clauses 37 to 76.
82. The method of clause 80 or clause 81, further comprising selecting an anti-cancer therapy for the individual in response to the cancer progression.
83. The method of clause 80 or clause 81, further comprising administering an anti-cancer therapy to the individual in response to the cancer progression.
84. The method of clause 80 or clause 81, further comprising adjusting an anti-cancer therapy for the individual in response to the cancer progression.
85. The method of any one of clauses 82 to 84, further comprising adjusting a dosage of the anti-cancer therapy or selecting a different anti-cancer therapy in response to the cancer progression.
86. The method of clause 85, further comprising administering the adjusted anti-cancer therapy to the individual.
87. The method of any one of clauses 80 to 86, wherein the first time point is before the individual has been administered an anti-cancer therapy, and wherein the second time point is after the individual has been administered the anti-cancer therapy.
88. The method of any one of clauses 80 to 87, wherein the individual has a cancer, is at risk of having a cancer, is being routine tested for cancer, or is suspected of having a cancer.
89. The method of any one of clauses 80 to 88, wherein the cancer is a solid tumor.
90. The method of any one of clauses 80 to 88, wherein the cancer is a hematological cancer.
91. The method of any one of clauses 82 to 90, wherein the anti-cancer therapy comprises chemotherapy, radiation therapy, immunotherapy, a targeted therapy, or surgery.
92. The method of any one of clauses 37 to 76, further comprising determining, identifying, or applying the mutational signature of the sample as a diagnostic value associated with the sample.
93. The method of any one of clauses 37 to 76, further comprising generating a genomic profile for the individual based on the determination of the mutational signature.
94. The method of clause 93, wherein the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.
95. The method of clause 93 or 94, wherein the genomic profile for the individual further comprises results from a nucleic acid sequencing-based test.
96. The method of any one of clauses 93 or 95, further comprising selecting an anti-cancer therapy, administering an anti-cancer therapy, or applying an anti-cancer therapy to the individual based on the generated genomic profile.
97. The method of any one of clauses 37 to 76, wherein the determination of the mutational signature associated with the sample is used in making suggested treatment decisions for the individual.
98. The method of any one of clauses 37 to 76, wherein the determination of the mutational signature associated with the sample is used in applying or administering a treatment to the individual.
99. A system comprising:
   one or more processors; and
   a memory communicatively coupled to the one or more processors and configured to store instructions that, when executed by the one or more processors, cause the system to perform a method comprising:
      receiving, using the one or more processors, sequence read data associated with the sample;
      determining, using the one or more processors, a mutational profile of the sample based on the sequence read data;
      inputting, using the one or more processors, the mutational profile into an encoder model, wherein the encoder model is trained using training data related to a plurality of mutational signatures; and
      predicting, using the one or more processors, one or more mutational signatures of the plurality of mutational signatures associated with the sample based on an output of the encoder model,
      wherein the output of the encoder model is associated with a dimensionality value, and wherein the dimensionality value is less than a number of the plurality of mutational signatures.
100. A non-transitory computer-readable storage medium storing one or more programs, the one or more programs comprising instructions, which when executed by one or more processors of a system, cause the system to perform a method comprising:
   receiving, using the one or more processors, sequence read data associated with the sample;
   determining, using the one or more processors, a mutational profile of the sample based on the sequence read data;
   inputting, using the one or more processors, the mutational profile into an encoder model, wherein the encoder model is trained using training data related to a plurality of mutational signatures; and
   predicting, using the one or more processors, one or more mutational signatures of the plurality of mutational signatures associated with the sample based on an output of the encoder model,
   wherein the output of the encoder model is associated with a dimensionality value, and wherein the dimensionality value is less than a number of the plurality of mutational signatures.
101. A method for identifying mutational signatures, the method comprising:
   receiving, using one or more processors, sequence read data associated with a plurality of samples;
   determining, using the one or more processors, mutational profiles of the plurality of samples based on the sequence read data;
   inputting, using the one or more processors, the mutational profiles into an encoder model; and
   inputting, using the one or more processors, outputs of the encoder model corresponding to the mutational profiles into an unsupervised machine learning model; and
   identifying, using the one or more processors, one or more mutational signatures based on clustering of the outputs of the encoder model.
102. The method of clause 101, wherein the unsupervised machine learning model comprises at least one of a density-based spatial clustering of applications with noise (DBSCAN), ordering points to identify the clustering structure (OPTICS); a gaussian mixture model (GMM), a k-means clustering model, a k nearest neighbors model, or a hierarchical clustering model.
103. The method of any of clauses 101 to 102, wherein a mutational profile of the mutational profiles comprise at least three mutations.
104. The method of any of clauses 101 to 103, wherein the mutational profiles are based on a targeted sequencing panel or a targeted-exome sequencing panel, a whole exome sequencing technique or a whole genome sequencing technique.
105. The method of any of clauses 101 to 104, wherein an output of the outputs of the encoder model is associated with a dimensionality value, and wherein the dimensionality value is less than a number of a plurality of mutational signatures used to obtain the output.
106. The method any of clauses 101 to 105, wherein the sequence read data associated with the plurality of samples is derived from single cell sequencing.
107. The method any of clauses 101 to 106, wherein the sequence read data associated with the plurality of samples is derived from circulating tumor DNA in a liquid biopsy sample.
108. The method any of clauses 101 to 107, wherein the sequence read data associated with the plurality of samples is derived from RNA in a liquid biopsy sample.
109. The method of any of clauses 101 to 108, wherein the mutational profiles comprise single-base substitutions.
110. The method of any of clauses 101 to 109, wherein the mutational profiles comprise double-base substitutions, insertions, deletions, copy number, rearrangement information, or a combination thereof.
111. The method of any of clauses 101 to 110, wherein the encoder model is trained by:
   receiving, using the one or more processors, the training data based on a plurality of training samples; and
   training, using the one or more processors, an autoencoder model comprising the encoder model based on the training data to obtain a trained autoencoder model.
112. The method of clause 111, wherein training the autoencoder model comprises:
   inputting, using the one or more processors, the training data into an untrained encoder model;
   inputting, using the one or more processors, an output of the untrained encoder model into a decoder model of the autoencoder model;
   determining, using the one or more processors, a reconstruction value of an output of the decoder model based on the training data and the output of the decoder model; and
   updating, using the one or more processors, the untrained encoder model based on the reconstruction value to produce a trained encoder model.
113. The method of any of clauses 111 to 112, wherein the training data comprises training mutational profiles comprising single-base substitutions, double-base substitutions, insertions, deletions, or a combination thereof.
114. The method of any of clauses 111 to 113, wherein the training data corresponds to a sequence length of 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, or a combination thereof.
115. The method of any of clauses 111 to 113, wherein the training data corresponds to a sequence length less than 0.5 Megabases.
116. The method of any of clauses 111 to 113, wherein the training data corresponds to a sequence length greater than 2.0 Megabases.
117. The method of any of clauses 111 to 116, further comprising:
   determining a number of mutations present in a training sample of the plurality of training samples;
   removing the training sample from the training data if the number of mutations present in the training sample is below a predetermined threshold.
118. The method of clause 117, wherein the predetermined threshold is greater than three.
119. The method of any of clauses 111 to 118, further comprising validating the trained autoencoder model, the validating comprising:
   obtaining, using the one or more processors, validation data based on the plurality of training samples, wherein the training data comprises the validation data;
   inputting, using the one or more processors, the validation data into the trained encoder model of the trained autoencoder model;
   inputting, using the one or more processors, an output of the trained encoder model into the decoder model;
   determining, using the one or more processors, a reconstruction value of a validation output of the decoder model based on the validation data; and
   updating, using the one or more processors, the encoder model based on the reconstruction value.
120. The method of any of clauses 111 to 118, further comprising validating the trained autoencoder model, the validating comprising:
   obtaining, using the one or more processors, validation data based on the plurality of training samples, wherein the training data comprises the validation data; and
   inputting, using the one or more processors, the validation data into the trained encoder model;
   inputting, using the one or more processors, an output of the trained encoder model into the decoder model;
   determining, using the one or more processors, a concordance value between the training data and an output of the decoder model; and
   updating, using the one or more processors, the encoder model based on the concordance value.
121. The method of any one of clauses 119 to 120, wherein obtaining the validation data comprises:
   receiving, by the one or more processors, raw validation data;
   down sampling, using the one or more processors, the raw validation data to obtain the validation data.
122. The method of clause 121, wherein the raw validation data comprises germline alterations and somatic alterations and the validation data comprises the somatic alterations.
123. The method of clause 122, wherein determining the reconstruction value is based on the somatic alterations comprising the raw validation data and the somatic alterations comprising the validation data.
124. The method of any of clauses 119 to 123, wherein the validation data is associated with sequence lengths of 0.5 Megabase, 0.6 Megabase, 0.7 Megabase, 0.8 Megabase, 0.9 Megabase, 1.0 Megabase, 1.1 Megabases, 1.2 Megabases, 1.3 Megabases, 1.4 Megabases, 1.5 Megabases, 1.6 Megabases, 1.7 Megabases, 1.8 Megabases, 1.9 Megabases, 2.0 Megabases, or a combination thereof.
125. The method of any of clauses 119 to 123, wherein the training data corresponds to a sequence length less than 0.5 Megabases.
126. The method of any of clauses 119 to 123, wherein the training data corresponds to a sequence length greater than 2.0 Megabases.
127. The method any of clauses 101 to 126, further comprising assigning, using the one or more processors, a therapy for the individual based on the one or more mutational signatures.
128. The method any of clauses 101 to 127, further comprising administering, using the one or more processors, a treatment to the individual based on the one or more mutational signatures.
129. The method any of clauses 101 to 128, further comprising associating, using the one or more processors, the individual with a clinical trial based on the one or more mutational signatures.
130. The method any of clauses 101 to 129, further comprising monitoring, using the one or more processors, a prognosis of the individual based on the one or more mutational signatures.
131. The method any of clauses 101 to 130, further comprising predicting, using the one or more processors, one or more clinical outcomes based on the one or more mutational signatures.
132. A method for diagnosing a disease, the method comprising: diagnosing that an individual has a disease based on a determination of a mutational signature associated with a sample from the individual, wherein the mutational signature is determined according to the method of any one of clauses 101 to 131.
133. A method of selecting an anti-cancer therapy, the method comprising: responsive to determining a mutational signature associated with sample from an individual, selecting an anti-cancer therapy for the individual, wherein mutational signature is determined according to the method of any one of clauses 101 to 131.
134. A method of treating a cancer in an individual, comprising: responsive to determining a mutational signature associated with a sample from an individual, administering an effective amount of an anti-cancer therapy to the individual, wherein the mutational signature is determined according to the method of any one of clauses 101 to 131.
135. A method for monitoring cancer progression or recurrence in an individual, the method comprising:
   determining a first mutational signature in a first sample obtained from the individual at a first time point according to the method of any one of clauses 101 to 131;
   determining a second mutational signature in a second sample obtained from the individual at a second time point; and
   comparing the first mutational signature to the second mutational signature, thereby monitoring the cancer progression or recurrence.
136. The method of clause 80, wherein the second mutational signature for the second sample is determined according to the method of any one of clauses 101 to 131.
137. The method of clause 80 or clause 81, further comprising selecting an anti-cancer therapy for the individual in response to the cancer progression.
138. The method of clause 80 or clause 81, further comprising administering an anti-cancer therapy to the individual in response to the cancer progression.
139. The method of clause 80 or clause 81, further comprising adjusting an anti-cancer therapy for the individual in response to the cancer progression.
140. The method of any one of clauses 82 to 84, further comprising adjusting a dosage of the anti-cancer therapy or selecting a different anti-cancer therapy in response to the cancer progression.
141. The method of clause 85, further comprising administering the adjusted anti-cancer therapy to the individual.
142. The method of any one of clauses 80 to 86, wherein the first time point is before the individual has been administered an anti-cancer therapy, and wherein the second time point is after the individual has been administered the anti-cancer therapy.
143. The method of any one of clauses 80 to 87, wherein the individual has a cancer, is at risk of having a cancer, is being routine tested for cancer, or is suspected of having a cancer.
144. The method of any one of clauses 80 to 88, wherein the cancer is a solid tumor.
145. The method of any one of clauses 80 to 88, wherein the cancer is a hematological cancer.
146. The method of any one of clauses 82 to 90, wherein the anti-cancer therapy comprises chemotherapy, radiation therapy, immunotherapy, a targeted therapy, or surgery.
147. The method of any one of clauses 101 to 131, further comprising determining, identifying, or applying the mutational signature of the sample as a diagnostic value associated with the sample.
148. The method of any one of clauses 101 to 131, further comprising generating a genomic profile for the individual based on the determination of the mutational signature.
149. The method of clause 148, wherein the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.
150. The method of any one of clauses 147 to 148, wherein the genomic profile for the individual further comprises results from a nucleic acid sequencing-based test.
151. The method of any one of clauses 147 to 150, further comprising selecting an anti-cancer therapy, administering an anti-cancer therapy, or applying an anti-cancer therapy to the individual based on the generated genomic profile.
152. The method of any one of clauses 101 to 131, wherein the determination of the mutational signature associated with the sample is used in making suggested treatment decisions for the individual.
153. The method of any one of clauses 101 to 131, wherein the determination of the mutational signature associated with the sample is used in applying or administering a treatment to the individual.
154. A system comprising:
   one or more processors; and
   a memory communicatively coupled to the one or more processors and configured to store instructions that, when executed by the one or more processors, cause the system to perform a method comprising:
      receiving, using the one or more processors, sequence read data associated with a plurality of samples;
      determining, using the one or more processors, mutational profiles of the plurality of samples based on the sequence read data;
      inputting, using the one or more processors, the mutational profiles into an encoder model; and
      inputting, using the one or more processors, outputs of the encoder model corresponding to the mutational profiles into an unsupervised machine learning model; and
      identifying, using the one or more processors, one or more mutational signatures based on clustering of the outputs of the encoder model.
155. A non-transitory computer-readable storage medium storing one or more programs, the one or more programs comprising instructions, which when executed by one or more processors of a system, cause the system to perform a method comprising:
   receiving, using the one or more processors, sequence read data associated with a plurality of samples;
   determining, using the one or more processors, mutational profiles of the plurality of samples based on the sequence read data;
   inputting, using the one or more processors, the mutational profiles into an encoder model; and
   inputting, using the one or more processors, outputs of the encoder model corresponding to the mutational profiles into an unsupervised machine learning model; and
   identifying, using the one or more processors, one or more mutational signatures based on clustering of the outputs of the encoder model.
156. A method for training an autoencoder model to predict a mutational profile of a sample, the method comprising:
   receiving, using one or more processors, training data comprising a plurality of mutational profiles from a plurality of training samples;
   inputting, using the one or more processors, the plurality of mutational profiles into an encoder model of the autoencoder model, wherein the encoder model is configured to output derived data;
   inputting the derived data into a decoder model of the autoencoder model, wherein the decoder model is configured to output a reconstruction of the plurality of mutational profiles based on the derived data;
   receiving a reconstruction value based on a comparison of the training data and the output of the decoder model; and
   updating the encoder model based on the reconstruction value to produce a trained encoder model.
157. The method of clause 156, further comprising validating the trained autoencoder model, the validating comprising:
   obtaining, using the one or more processors, validation data based on the plurality of training samples; and
   inputting, using the one or more processors, the validation data into the trained encoder model of the autoencoder model;
   inputting, using the one or more processors, an output of the trained encoder model into the decoder model;
   determining, using the one or more processors, a reconstruction value of an output of the decoder model based on the validation data; and
   updating, using the one or more processors, the encoder model based on the reconstruction value.
158. The method of clause 156, further comprising validating the trained autoencoder model, the validating comprising:
   obtaining, using the one or more processors, validation data based on the plurality of training samples; and
   inputting, using the one or more processors, the validation data into the trained encoder model;
   inputting, using the one or more processors, an output of the trained encoder model into the decoder model;
   determining, using the one or more processors, a concordance value between the training data and an output of the decoder model; and
   updating, using the one or more processors, the encoder model based on the concordance value.
159. The method of any one of clauses 157 to 158, wherein obtaining the validation data comprises:
   receiving, by the one or more processors, raw validation data corresponding to one or more mutational profiles;
   down sampling, using the one or more processors, the raw validation data to obtain the validation data, and
   wherein the reconstruction value or the concordance value is determined based on the raw validation data.
160. A method of determining an alkylating agent resistance signature status of a sample from a subject, the method comprising:
   receiving, using one or more processors, sequence read data associated with a sample from the subject;
   selecting, using the one or more processors, a plurality of reads from the sequence read data;
   determining, using the one or more processors, an alkylating agent resistance profile based on the selected plurality of reads;
   inputting, using the one or more processors, the alkylating agent resistance profile into a statistical model; and
   predicting, using the one or more processors, the alkylating agent resistance signature status based on an output of the statistical model.
161. A method of treating a subject having a cancer, the method comprising:
   (a) determining an alkylating agent resistance signature status of a sample from the subject; and
   (b) treating the subject with an alkylating agent if the subject is determined to have a non-positive alkylating agent resistance signature status.
162. The method of clause 161, wherein determining an alkylating agent resistance signature status is based on the method of clause 160.
163. The method of clause 161, wherein the subject was previously treated with one or more alkylating agents.
164. The method of any one of clauses 160-163, further comprising determining a tumor mutation burden (TMB) in the sample from the subject.
165. The method of any one of clauses 160-164, wherein the statistical model comprises an encoder model as clauseed in clauses 37-76.
166. The method of any one of clauses 160-165, wherein the sample comprises a tissue biopsy sample, a liquid biopsy sample, or a normal control.
167. The method of clause 166, wherein the sample is a liquid biopsy sample and comprises blood, plasma, cerebrospinal fluid, sputum, stool, urine, or saliva.
168. The method of clause 167, wherein the sample is a liquid biopsy sample and comprises circulating tumor cells (CTCs), cell-free DNA (cfDNA), circulating tumor DNA (ctDNA), or any combination thereof.
169. The method any of clauses 160-168, wherein the sequence read data associated with the sample is derived from RNA in a liquid biopsy sample.
170. The method of any of clauses 160-169, wherein the alkylating agent resistance profile comprises single-base substitutions.
171. The method of any of clauses 160-170, wherein the alkylating agent resistance profile comprises double-base substitutions, insertions, deletions, copy number, rearrangement information, or a combination thereof.
172. The method of any of clauses 160-171, further comprising determining, based on the selected plurality of reads, a MSH6 alteration, a MSH2 alteration, a MLH1 alteration, a PIK3CA alteration, a TP53 alteration, or a combination thereof.
173. The method of any of clauses 161-172, wherein the cancer is a brain cancer, a breast cancer, a colorectal cancer (CRC), a prostate cancer, a non-small cell lung cancer (NSCLC), a neuroendocrine cancer, or a combination thereof.
174. A method for identifying mutational signatures associated with a sample from an individual, the method comprising:
   receiving, using one or more processors, sequence read data associated with the sample;
   determining, using the one or more processors, a mutational profile of the sample based on the sequence read data;
   inputting, using the one or more processors, the mutational profile into an encoder model, wherein the encoder model is trained using training data related to a plurality of mutational signatures; and
   predicting, using the one or more processors, one or more mutational signatures of the plurality of mutational signatures associated with the sample based on an output of the encoder model,
   wherein the output of the encoder model is associated with a dimensionality value, and wherein the dimensionality value is less than or equal to a number of the plurality of mutational signatures.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads;
determining, using the one or more processors, a mutational profile of the sample based on the sequence read data;
inputting, using the one or more processors, the mutational profile into an autoencoder model, wherein the autoencoder model is trained using training data related to a plurality of mutational signatures; and
predicting, using the one or more processors, one or more mutational signatures of the plurality of mutational signatures associated with the sample based on an output of the autoencoder model,
wherein the output of the autoencoder model is associated with a dimensionality value, and wherein the dimensionality value is less than a number of the plurality of mutational signatures.

2. The method of claim 1, wherein the plurality of mutational signatures corresponds to a plurality of predefined mutational signatures.

3. The method of claim 1, wherein the plurality of mutational signatures corresponds to a plurality of COSMIC mutational signatures.

4. The method of claim 1, wherein the plurality of predefined mutational signatures comprises a DNA mismatch repair (MMR) signature, a tobacco signature, a UV signature, a putative polymerase epsilon (POLE) signature, an apolipoprotein B mRNA-editing enzyme, catalytic polypeptide (APOBEC) signature, an alkylating agent resistance signature, or a combination thereof.

5. The method of claim 1, wherein predicting the one or more mutational signatures comprises inputting the output of the encoder model into a statistical model.

6. The method of claim 5, wherein the statistical model comprises at least one of a random forest model, a gradient boosting model, a logistic regression model, a support vector machine (SVM), a decision tree model, a density-based spatial clustering of applications with noise (DBSCAN), ordering points to identify the clustering structure (OPTICS); a gaussian mixture model (GMM), a k-means clustering model, a k nearest neighbors model, or a hierarchical clustering model.

7. The method of claim 5, further comprising training the statistical model, wherein the statistical model is a classifier model, wherein training the statistical model comprises:
inputting, using the one or more processors, a plurality of training mutational profiles into the encoder model;
inputting, using the one or more processors, outputs of the encoder model corresponding to the plurality of training mutational profiles into an unlabeled classifier model; and
identifying, using the one or more processors, one or more mutational signatures based on clustering of the outputs of the encoder model.

8. The method of claim 1, wherein the mutational profile comprises less than twenty mutations or at least three mutations.

9. The method of claim 1, wherein the mutational profile is based on a targeted-sequencing panel, a targeted-exome sequencing panel, a whole exome sequencing technique, or a whole genome sequencing technique.

10. The method of claim 1, wherein the sequence read data associated with the sample is derived from a biopsy sample.

11. The method of claim 1, wherein the mutational profile comprises single-base substitutions, double-base substitutions, insertions, deletions, copy number, rearrangement information, or a combination thereof.

12. The method of claim 1, wherein the encoder model is trained by:
receiving, using the one or more processors, the training data based on a plurality of training samples; and
training, using the one or more processors, an autoencoder model comprising the encoder model based on the training data to obtain a trained autoencoder model.

13. The method of claim 12, wherein training the autoencoder model comprises:
inputting, using the one or more processors, the training data into an untrained encoder model;
inputting, using the one or more processors, an output of the untrained encoder model into a decoder model of the autoencoder model;
determining, using the one or more processors, a reconstruction value of an output of the decoder model based on the training data and the output of the decoder model; and
updating, using the one or more processors, the untrained encoder model based on the reconstruction value.

14. The method of claim 1, wherein the training data comprises germline alterations and somatic alterations and the reconstruction value is based on the somatic alterations.

15. The method of claim 1, further comprising:
determining a number of mutations present in a training sample of the plurality of training samples;
removing the training sample from the training data if the number of mutations present in the training sample is below a predetermined threshold.

16. The method of claim 1, further comprising validating the trained autoencoder model, the validating comprising:
obtaining, using the one or more processors, validation data based on the plurality of training samples, wherein the training data comprises the validation data; and
inputting, using the one or more processors, the validation data into a trained encoder model of the trained autoencoder model;
inputting, using the one or more processors, an output of the trained encoder model into the decoder model;
determining, using the one or more processors, a reconstruction value of a validation output of the decoder model based on the validation data; and
updating, using the one or more processors, the encoder model based on the reconstruction value.

17. The method of claim 1, further comprising validating the trained autoencoder model, the validating comprising:
obtaining, using the one or more processors, validation data based on the plurality of training samples, wherein the training data comprises the validation data; and
inputting, using the one or more processors, the validation data into a trained encoder model of the trained autoencoder model;
inputting, using the one or more processors, an output of the trained encoder model into the decoder model;
determining, using the one or more processors, a concordance value between the training data and a validation output of the decoder model; and
updating, using the one or more processors, the encoder model based on the concordance value.

18. The method of claim 16, wherein obtaining the validation data comprises:
receiving, by the one or more processors, raw validation data;
down sampling, using the one or more processors, the raw validation data to obtain the validation data, and
wherein the reconstruction value or the concordance value is determined based on the raw validation data.

19. The method of claim 18, wherein determining the reconstruction value is based on the somatic alterations comprising the raw validation data and the somatic alterations comprising the validation data.

20. A method for identifying mutational signatures, the method comprising:
receiving, using one or more processors, sequence read data associated with a plurality of samples;
determining, using the one or more processors, mutational profiles of the plurality of samples based on the sequence read data;
inputting, using the one or more processors, the mutational profiles into an encoder model; and
inputting, using the one or more processors, outputs of the encoder model corresponding to the mutational profiles into an unsupervised machine learning model; and
identifying, using the one or more processors, one or more mutational signatures based on clustering of the outputs of the encoder model.
